(19) United States
(12) Patent Application Publication
(10) Pub. No.: US 2023/0056646 A1
(43) Pub. Date: Feb. 23, 2023

(54) RECOMBINANT PROTEINS HAVING ENZYME ACTIVITY AGAINST MICROCYSTIN AND METHODS OF WATER REMEDIATION

(71) Applicant: United States of America as Represented by The Secretary of The Army, Alexandria, VA (US)

(72) Inventors: Carina M Jung, Vicksburg, MS (US); Karl J Indest, Ridgeland, MS (US); Matthew R Carr, Madison, MS (US); Nathan E Harms, Edwards, MS (US)

(21) Appl. No.: 17/405,012

(22) Filed: Aug. 17, 2021

(51) Int. Cl. C12N 9/48; C02F 3/34

(52) U.S. Cl. CPC C12N 9/48; C02F 3/342; C02F 2101/38

(57) ABSTRACT

Recombinant MlrA and MlrB proteins having enzymatic activity against microcystin (MC) degrade and reduce the toxicity of MC. Compositions of the proteins can be used in the remediation of MC toxin generated from harmful cyanobacterial and algal blooms. Recombinant proteins, nucleic acids, host cells, and methods of producing the MlrA and MlrB are disclosed.

### GOVERNMENT RIGHTS

The subject matter of this disclosure was made with support from the United States Army Corps of Engineers—Engineer Research and Development Center, Aquatic Nuisance Species Research Program. The Government of the United States of America has certain rights in this invention.

### STATEMENT REGARDING SEQUENCE LISTING

The instant application contains a Sequence Listing which is hereby incorporated by reference in its entirety. The ASCII copy, created on Jul. 19, 2021, is named Microcystin_SEQ_LST and is 153 kbytes in size.

### FIELD

The present disclosure relates to the engineering and production of recombinant polypeptides having enzymatic activity against microcystin (MC) and the remediation of microcystin toxin generated from a harmful cyanobacterial/algal bloom (HAB). The enzymatic activity of the recombinant polypeptides acts on MC and analogues thereof to degrade and detoxify the MC.

### BACKGROUND

Harmful cyanobacterial/algal blooms are a worldwide problem due to their massive growth potential and their ability to clog waterways, physically impair aquatic wildlife movement, and inhibit oxygen exchange. Cyanobacteria containing toxins are of particular concern as they have been documented in almost all states and are a high priority concern for inland waterways (Erickson et al. 2016; Loftin et al. 2016). The United States Environmental Protection Agency estimates the economic impact of nutrients and HABs on tourism alone to be about $1 billion per year. Moreover, the issue of cyanobacterial HABs is expected to grow as agriculturally induced eutrophication and climate change scenarios predict that in the coming years, waterways will experience heightened conditions that favor cyanobacteria productivity (Paerl 2014). The ability to mitigate toxic bloom events quickly and without the use of harmful chemicals is a primary goal to ensure the safety of aquatic life and human health and allow authorities to safely manage the HAB biomass.

Some commonly occurring HAB forming cyanobacteria include the genera Microcystis, Anabaena, and Planktothrix (Oscillatoria), with microcystins (MCs) being the most reported toxins in freshwater (Saito et al. 2003; Yang et al. 2014). MCs are cyclic peptides and known hepatotoxins that can result in liver damage, heart failure, and death (Ozawa et al. 2003; Yang et al. 2014; WHO 2003). Over 100 MC variants have been identified to date, having the same basic structure (FIG. 1), where X and Y represent variable L-amino acids (Ozawa et al. 2003). While the MC variants have differing levels of toxicity, MC-LR is generally considered the most toxic, most common, and most closely linked to liver cancer and other diseases in humans and animals. MC-LR exerts its harmful effects by binding to type 1 and 2A protein phosphatases in the liver, resulting in excessive phosphorylation.

Remediation strategies are needed to degrade or inactivate MCs when a toxic event is suspected. Unfortunately, conventional methods for water treatment such as high temperatures, chlorination, extreme pH, and ultra-violet treatment, have proven to be expensive and less than successful at removing these toxins. There is a continuing need for efficient and cost-effective methods of MC removal from water.

Biological degradation of MCs by bacteria is one form of remediation that has not yet been fully utilized. Naturally occurring populations of bacteria have been shown to degrade MC toxins, most typically through the mlrABCD gene cluster (FIG. 2) (Massey and Yang 2020). An enzyme coded by the mlrA gene opens the cyclic MC structure by cleaving the ADDA-Arg peptide bond in microcystin LR (Saito et al. 2003), rendering the linearized MC up to 160 times less toxic (Lezcano et al. 2016). A second gene, mlrB, codes for a serine protease that further degrades the linearized MC into smaller peptides, facilitating more complete degradation. Additional peptidases, including but not limited to that encoded by mlrC, further degrade the linear MC structure (Saito et al. 2003), diminishing MC toxicity (FIG. 3) (Massey and Yang 2020).

Detoxification of MC by naturally occurring bacteria has been known in the art and numerous bacterial groups have been noted to contain versions of the mlr gene group (e.g., FIG. 4 and FIG. 5). However, the low naturally occurring MlrA and MlrB concentrations that may be present in waterways are not sufficient to successfully detoxify MC contaminated water.

A need exists to engineer and produce quantities of MC-degrading enzymes that can be effectively used to deactivate these harmful toxins on a large, field-level scale. The need also includes a shelf-stable enzyme composition that can be safely used in the field by cleanup personnel and provide a safe working environment.

The present disclosure engineers a synthetic recombinant DNA construct for use with microorganisms to generate large quantities of MC degrading enzymes for administration to HAB-affected waters.

### SUMMARY

The description herein discloses the engineering and production of recombinant proteins having enzymatic activity against microcystin (MC). The recombinant proteins include MlrA, MlrB, or a combination of both MlrA and MlrB, and according to various embodiments, the proteins have MlrA enzyme activity, MlrB enzyme activity, or a combination of MlrA and MlrB enzyme activity against MC. The MlrA and MlrB enzyme proteins utilize MC as a substrate and their enzyme activity, both individually and collectively, degrade and detoxify MC. MlrA and MlrB work in concert as the degradation product of MlrA is the substrate for MlrB.

The present description also discloses a composition that contains a recombinant protein having enzymatic activity against MC. The composition contains one or more recombinant protein that includes MlrA, MlrB, or a combination of both MlrA and MlrB, and according to various embodiments, the composition has MlrA enzyme activity, MlrB enzyme activity, or a combination of both MlrA and MlrB enzyme activities. The composition degrades and detoxifies MC.

The present specification also discloses a recombinant nucleic acid encoding one or more proteins having enzymatic activity against MC. According to various embodiments, the nucleic acid encodes for MlrA, MlrB, or for both MlrA and MlrB.

The present specification further discloses methods of degrading MC that include contacting the MC with a recombinant protein containing MlrA, MlrB, or a combination of MlrA and MlrB, the protein having MlrA enzyme activity, MlrB enzyme activity, or a combination of MlrA and MlrB enzyme activity against the microcystin.

The present specification further discloses methods of treating water contaminated by a HAB. The contaminated water contains MC, and the methods reduce the level of and/or detoxify the MC in the MC-contaminated water. The methods include bringing the water into contact with an effective amount of a recombinant protein containing MlrA, MlrB, or a combination of MlrA and MlrB, the protein having MlrA enzyme activity, MlrB enzyme activity, or a combination of MlrA and MlrB enzyme activity against the microcystin.

The present specification further discloses a recombinant cell containing a heterologous gene encoding a recombinant protein having enzyme activity against MC. The recombinant protein includes MlrA, MlrB, or both MlrA and MlrB, and according to various embodiments, the protein has MlrA enzyme activity, MlrB enzyme activity, or a combination of MlrA and MlrB enzyme activity.

The present specification further discloses a method of producing a recombinant protein having enzyme activity against MC. The method includes culturing a recombinant microorganism in a culture medium, the microorganism containing a heterologous mlrA, mlrB, or both mlrA and mlrB gene encoding the protein(s) having enzyme activity against MC.

The present specification also provides a biofilter containing a medium and a biofilm on the medium, wherein the biofilm is formed from a group of cells that includes the recombinant cells as disclosed herein containing a heterologous gene encoding a recombinant protein having enzyme activity against MC, wherein the recombinant cells are capable of degrading MC.

The present specification further provides a method of filtering water to remove MC by passing the water through a biofilter that contains a medium and a biofilm on the medium, wherein the biofilm is formed from a group of cells that includes recombinant cells as disclosed herein containing a heterologous gene encoding a recombinant protein having enzyme activity against MC, wherein the recombinant cells are capable of degrading MC.

### BRIEF DESCRIPTION OF THE DRAWINGS

The objects, features and advantages of the present disclosure will become more apparent from the following detailed description, which proceeds with reference to the accompanying figures.

FIG. 1 shows the chemical structure of microcystin-LR and microcystin-RR. Microcystins primarily differ in the two amino acids indicated as X and Y (Ozawa et al. 2003).

FIG. 2 is a schematic illustrating the mlrABCD gene cluster in Sphingopyxis sp. C-1 as viewed with Geneious Prime software (Biomatters, Inc., San Diego, Calif.)

FIG. 3 is a schematic showing an enzymatic degradation pathway of MC-LR by mlrA and mlrB (Massey and Yang 2020).

FIG. 4A-4E is a DNA sequence alignment of mlrA from Sphingopyxis sp. C-1 (Genbank Accession #B468058) (SEQ ID NO: 1) to a variety of other organisms. Sphingomonas sp. NV3 (JN256930) (SEQ ID NO: 10), Sphingomonas sp. USTB-05 (HM245411) (SEQ ID NO: 12), Novosphingobium sp. THN1 (CP028347) (SEQ ID NO: 14), Acinetobacter lwoffii strain A6 (KU977292) (SEQ ID NO: 16), Stenotrophomonas sp. EMS (GU224277) (SEQ ID NO: 18), Catellibacterium terrae strain A2 (KU977291) (SEQ ID NO: 20), Kurthia gibsonii strain A1 (KU977290) (SEQ ID NO: 22), Rhizobium sp. TH (KX371892) (SEQ ID NO: 24), Bacillus cereus strain Q1 (KU977293) (SEQ ID NO: 26).

FIG. 5A-5E is a DNA sequence alignment of mlrB from Sphingopyxis sp. C-1 (Genbank accession #AB468059) (SEQ ID NO: 3) to a variety of other organisms. Sphingomonas sp. USTB-05 (KC513423) (SEQ ID NO: 28), Novosphingobium sp. THN1 (CP028347) (SEQ ID NO: 30), Sphingosinicella microcystinivorans B9 (AP018711) (SEQ ID NO: 32), and Rhizobium sp. TH (KX371892) (SEQ ID NO: 34).

FIG. 6 is a DNA sequence alignment between the Sphingopyxis sp. C-1 mlrA gene (Genbank accession #AB468058) (SEQ ID NO: 1) and a codon optimized version for expression in E. coli (SEQ ID NO: 2).

FIGS. 7A and 7B are a DNA sequence alignment between the Sphingopyxis sp. C-1 mlrB gene (Genbank accession #AB468059) (SEQ ID NO: 3) and a codon optimized version for expression in E. coli (SEQ ID NO: 4).

FIG. 8 shows a plasmid map of pET-21a_mlrA, which is an embodiment that includes codon optimized mlrA cloned in pET-21a (SEQ ID NO: 7).

FIG. 9 shows a plasmid map of pET-21a_mlrB, which is an embodiment that includes codon optimized mlrB cloned in pET-21a (SEQ ID NO: 8).

FIG. 10 shows a plasmid map of pET-21_mlrA_mlrB, which is an embodiment that includes a bicistronic arrangement of codon optimized mlrA and mlrB cloned in pET-21a (SEQ ID NO: 9).

FIGS. 11A and 11B are graphs respectively showing (A) the degradation of native circular microcystin and (B) the production of linear microcystin over time from E. coli cultures expressing mlrA, mlrB, mlrA+mlrB, or mlrAB.

FIG. 12 is a graph presenting a growth curve of the E. coli mlrAB strain and BL21 control strain in minimal medium M9.

FIGS. 13A, 13B, and 13C are graphs respectively showing (A) the degradation of native circular microcystin and (B) the accumulation of MC breakdown products linear MC and (C) tetrapeptide over time from E. coli cultures expressing mlrAB.

FIG. 14 is a graph showing the degradation of MC by cell free (filtered) medium used to grow induced (IPTG) and uninduced E. coli mlrA and mlrAB strains, along with a BL21 control strain.

FIG. 15 is a graph showing MC degradation by filter concentrated (50×) crude protein from the culture of E. coli mlrAB strain.

### DETAILED DESCRIPTION

While the present disclosure will be described in conjunction with embodiments, the objects, features, and advantages of the disclosure can be applied to a wide variety of applications, and the description herein is intended to cover alternatives, modifications, and equivalents within the spirit and scope of the disclosure and the claims. The description in the present disclosure should not be viewed as limiting or as setting forth the only embodiments as the disclosure encompasses other embodiments not specifically recited in this description.

Throughout the present specification and the accompanying claims, the words “comprise”, “include”, “contain, and “having” and variations such as “comprises”, “comprising”, “includes”, “including” and “containing” are to be interpreted inclusively. That is, these words are intended to convey the possible inclusion of other elements or integers not specifically recited, where the context allows.

The articles “a” and “an” are used herein to refer to one or to more than one of the grammatical object of the article. By way of example, “an element” may mean one element or more than one element.

As used herein, the terms “polynucleotide” and “nucleic acid” are used interchangeably and each refers to a single- or double-stranded polymer of deoxyribonucleotide bases, ribonucleotide bases, known analogues of natural deoxyribonucleotide bases and ribonucleotide bases, or mixtures thereof. The terms include reference to the specified sequence as well as to the sequence complimentary thereto, unless otherwise indicated.

As used herein, the terms “protein” and “polypeptide” are used interchangeably and each refers to a polymer made up of amino acids linked together by peptide bonds.

The term “recombinant” as used herein indicates that the material (e.g., a nucleic acid or a polypeptide) has been artificially or synthetically altered by human intervention. For example, a “recombinant polynucleotide” or “recombinant nucleic acid” as used herein refers to a polynucleotide or nucleic acid that is not in its native state. For example, the nucleotide sequence at issue can be cloned into a vector, or otherwise combined with one or more additional nucleic acids. The term “recombinant polypeptide” or “recombinant protein” as used herein refers to a protein molecule that is expressed using a recombinant nucleic acid molecule. A “recombinant cell” or “recombinant host cell” refers to a cell into which exogenous (non-native) genetic material has been introduced, or a cell that contains and/or expresses a recombinant nucleic acid or recombinant polynucleotide.

The term “percent identity” as used herein refers to a relationship between two or more polypeptide sequences or two or more polynucleotide sequences, as determined by comparing the sequences. Percent identity can be calculated by known methods using a sequence alignment program.

BLASTN may be used to identify a nucleic acid sequence having at least 70%, 75%, 80%, 85%, 87.5%, 90%, 92.5%, 95%, 97.5%, 98%, 99%, or any percent identity to a reference nucleic acid. BLASTP may be used to identify an amino acid sequence having at least 70%, 75%, 80%, 85%, 87.5%, 90%, 92.5%, 95%, 97.5%, 98%, 99% or any percent identity to a reference amino acid sequence. Various default settings for BLASTN and BLASTP are described by and incorporated by reference to the disclosure available at the U.S. National Library of Medicine, National Center for Biological Medicine and available on its website.

As used herein, a “vector” refers to any means by which a nucleic acid can be propagated and/or transferred between different host cells. Vectors include viruses, bacteriophage, plasmids, viral vectors, expression vectors, gene transfer vectors, minicircle vectors, artificial chromosomes, and the like. Vectors can be “episomes,” that is they replicate autonomously, or can integrate into a chromosome of a host cell.

As used herein, an “expression vector” refers to a recombinant DNA molecule containing a desired coding sequence and appropriate nucleic acid sequences for the expression of an operably linked coding sequence in a particular host cell. Nucleic acid sequences for expression in prokaryotes typically include a promoter, an operator sequence, a ribosome binding site, and possibly other sequences. A secretory signal peptide sequence can also be encoded by the expression vector, operably linked to the desired coding sequence so that the expressed protein can be secreted by the recombinant host cell, for more facile isolation of the protein from the cell.

As used herein, the term “operably linked” refers to a configuration in which a control sequence is appropriately placed (i.e., in a functional relationship) at a position relative to a nucleic acid sequence of interest such that the control sequence directs or regulates the expression of the nucleic acid and/or polypeptide of interest. For example, a promoter is operably linked with a coding sequence when it can affect the expression of that coding sequence, i.e., the coding sequence is under the transcriptional control of the promoter. In general, a control sequence includes, but is not limited to, promoter sequences, ribosomal binding sites, transcriptional start and stop sequences, translational start and stop sequences, and enhancer or activator sequences.

The term “codon optimized” or “codon optimization” as used herein refers to the alteration of codons in the gene or coding regions of the nucleic acid to reflect the typical codon usage of the host organism without altering the polypeptide encoded by the DNA. Such optimization includes replacing at least one, or more than one, or a significant number of, codons with one or more codons that are more frequently used in the genes of the host organism. Codon optimization can be determined by various methods known in the art, such as with codon usage tables or using the Geneious Prime software (Biomatters, Inc., San Diego, Calif.), or OPTIMUMGENE™ codon optimization algorithm (GENSCRIPT®, Piscataway, N.J.).

As used herein, the terms “gene” or “coding sequence” refer to the nucleic acid sequence that is transcribed and translated into a polypeptide. The gene may or may not include regions preceding and following the coding region, e.g., 5′ untranslated or leader sequences and 3′ untranslated or trailer sequences. A “heterologous” gene refers to a gene not normally found in the host cell but that is introduced into the host cell by gene transfer.

A “cistron” refers to a segment of DNA or RNA that codes for a specific polypeptide. As used herein, the term “bicistronic” refers to the existence in a recombinant nucleic acid of two cistrons that are expressed from a single transcriptional unit. Thus, in bicistronic nucleic acid, a single mRNA transcript contains two coding regions. For example, a first cistron contains an open reading frame encoding a first polypeptide, such as a first enzyme, while a second cistron contains an open reading frame encoding a second polypeptide, such as a second enzyme.

As used herein, the term “expression” includes any step involved in the production of a polypeptide or protein including, but not limited to, transcription, post-transcriptional modification, translation, post-translational modification, and secretion. Generally, expression includes the transcription, i.e., the synthesis of a mRNA based on the DNA sequence of the gene, and the translation of the mRNA into the corresponding polypeptide chain, which may additionally be modified post-translationally.

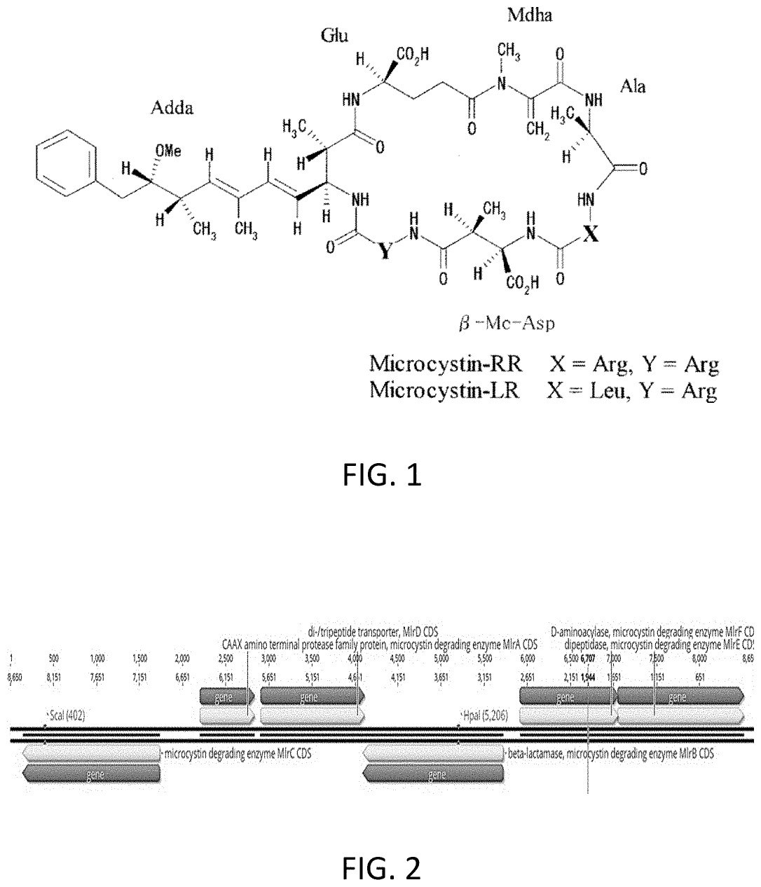

The term “microcystin” or “MC” as used herein refers to a class of toxins produced by certain freshwater cyanobacteria, such as Microcystis aeruginosa and other Microcystis species, as well as members of the Planktothrix, Anabaena, Fischerella, Gloeotrichia, Nodularia, Oscillatoriaxi, and Nostoc genera. Chemically, MCs are cyclic heptapeptides with a general structure of cyclo-(D-alanine¹-X²-D-MeAsp³-Y⁴-Adda⁵-D-glutamate⁶-Mdha⁷), in which X and Y are variable L-amino acids. The main isoforms are exemplified by MC-RR and MC-LR (FIG. 1).

As used herein, the term “degrade microcystin” or “degrade MC” refers to degradation or breaking down of MC into smaller components and the conversion of MC to a form that has reduced toxicity compared to the starting compound.

The term “enzyme activity” or “enzymatic activity” as used herein refers to the general catalytic properties of an enzyme and a chemical process in which an enzyme catalyzes conversion of one or more molecules into different molecules.

As used herein, “MlrA” and “MlrB” refer to polypeptides or proteins, while “mlrA” and “mlrB” refer to genes respectively encoding MlrA and MlrB, and “mlrAB” refers to genes encoding both MlrA and MlrB in a bicistronic arrangement. The terms “mlrA strain”, “mlrB strain”, and “mlrAB strain” refer to recombinant cells, such as E. coli, containing heterologous genes for mlrA, mlrB and mlrAB, respectively.

A first aspect of the present specification discloses the engineering and production of recombinant proteins having enzymatic activity against microcystin (MC). The recombinant proteins include MlrA, MlrB, or both MlrA and MlrB, and according to various embodiments, the recombinant proteins have MlrA enzyme activity, MlrB enzyme activity, or a combination of MlrA and MlrB enzyme activity against MC. The MlrA and MlrB enzymes utilize MC as a substrate and their enzyme activity, both individually and collectively, degrade and detoxify MC.

According to various embodiments of the recombinant proteins, at least one of the MlrA and MlrB proteins is from a microorganism selected from Sphingopyxis, Sphingomona, Novosphingobium, Acinetobacter, Sphingosinicella, Stenotrophomonas, Ca tellibacterium, Kurthia, Rhizobium, Phyllobacterium, Actinoplanes, Pseudoxanthomonas, or Bacillus. In some embodiments, at least one of the MlrA and MlrB proteins is from Sphingopyxis sp. C-1. In some embodiments, both the MlrA and MlrB proteins are from Sphingopyxis sp. C-1.

According to various embodiments, the recombinant MlrA protein has the amino acid sequence of SEQ ID NO: 5, or has at least 70% identity to the amino acid sequence of SEQ ID NO: 5 and has enzymatic activity against cyclic MC. Various embodiments of the recombinant MlrA protein have at least 75%, 80%, 85%, 90%, 95%, or 98% identity to the amino acid sequence of SEQ ID NO: 5 and have enzymatic activity against cyclic MC.

According to various embodiments, the recombinant MlrA protein has the amino acid sequence selected from SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, or SEQ ID NO: 27, or has at least 70% identity to the amino acid sequence of SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, or SEQ ID NO: 27 and has enzymatic activity against cyclic MC. Various embodiments of the recombinant MlrA protein have at least 75%, 80%, 85%, 90%, 95%, or 98% identity to the amino acid sequence of SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, or SEQ ID NO: 27 and have enzymatic activity against cyclic MC.

According to various embodiments, the recombinant MlrB protein has the amino acid sequence of SEQ ID NO: 6, or has at least 70% identity to the amino acid sequence of SEQ ID NO: 6 and has enzymatic activity against linearized MC. Various embodiments of the recombinant MlrB protein have at least 75%, 80%, 85%, 90%, 95%, or 98% identity to the amino acid sequence of SEQ ID NO: 6 and have enzymatic activity against linearized MC.

According to various embodiments, the recombinant MlrB protein has the amino acid sequence selected from SEQ ID NO: 29, SEQ ID NO: 31, SEQ ID NO: 33, or SEQ ID NO: 35, or has at least 70% identity to the amino acid sequence of SEQ ID NO: 29, SEQ ID NO: 31, SEQ ID NO: 33, or SEQ ID NO: 35 and has enzymatic activity against linearized MC. Various embodiments of the recombinant MlrB protein have at least 75%, 80%, 85%, 90%, 95%, or 98% identity to the amino acid sequence of SEQ ID NO: 29, SEQ ID NO: 31, SEQ ID NO: 33, or SEQ ID NO: 35 and have enzymatic activity against linearized MC.

Chemically, microcystins are cyclic heptapeptides. The seven amino acids that are involved in the structure of a microcystin include a unique β-amino acid (ADDA), along with alanine (D-ala), D-β-methyl-isoaspartate (D-β-Me-isoAsp), and glutamic acid (D-glu). Furthermore, microcystins contain two variable residues, which provides the differentiation between variants of microcystins. For instance, in microcystin-LR the two variable residues are leucine and arginine; in microcystin-RR they are arginine and arginine (FIG. 1). Over one hundred microcystins have been identified to date, representing differences in the two variable residues and some modifications in the other amino acids. Different microcystins have different toxicity profiles, with microcystin-LR generally recognized to be the most toxic.

According to various embodiments, the recombinant protein has MlrA enzyme activity, MlrB enzyme activity, or a combination of MlrA and MlrB enzyme activity against a variety of MCs, such as Microcystin-LR, Microcystin-RR, Microcystin-YR, Microcystin-LA, Microcystin-LY, Microcystin-LW, and Microcystin-LF. The recombinant protein has enzymatic activity against MC and can detoxify MC produced by a variety of cyanobacteria, such as Microcystis, Planktothrix, Anabaena, Fischerella, Gloeotrichia, Nodularia, Oscillatoriaxi, and Nostoc.

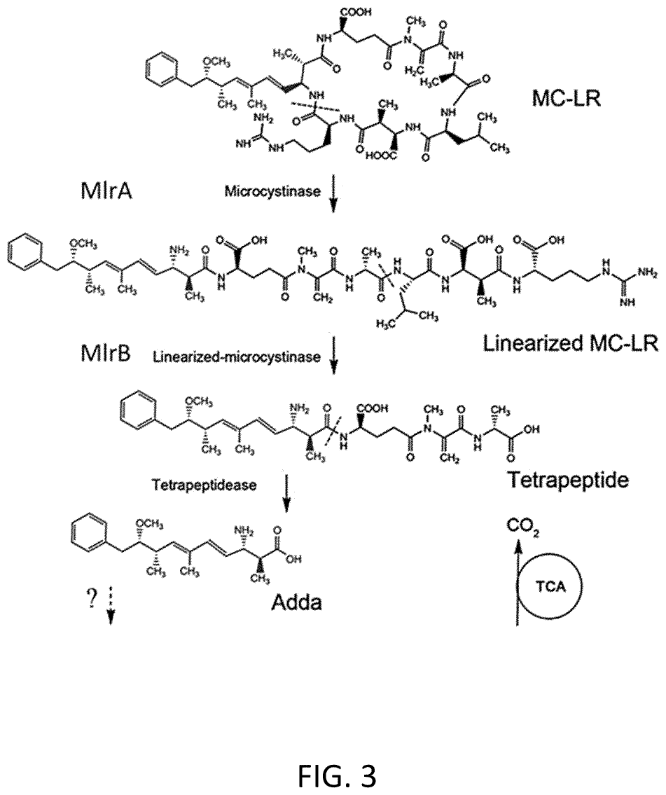

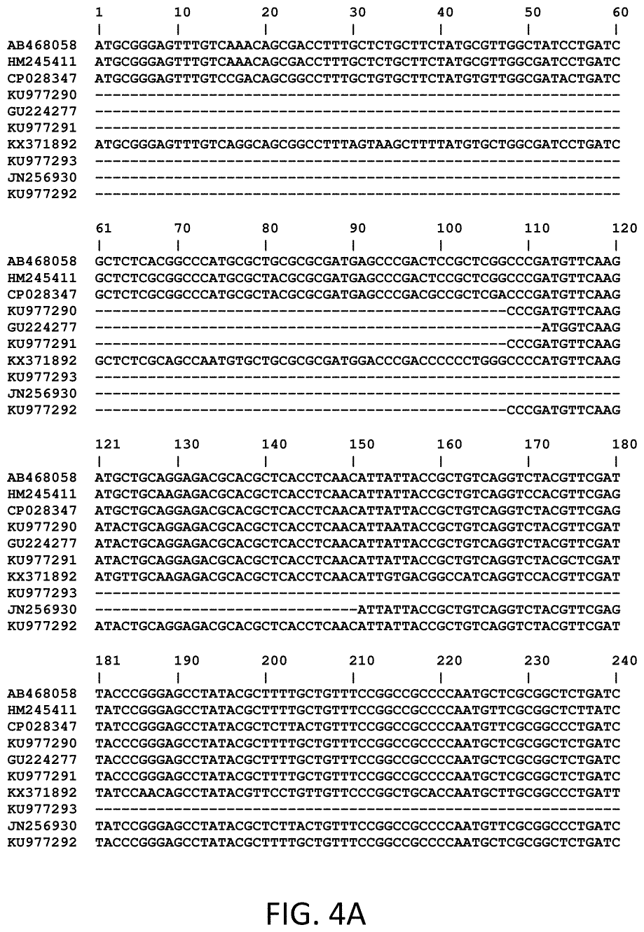

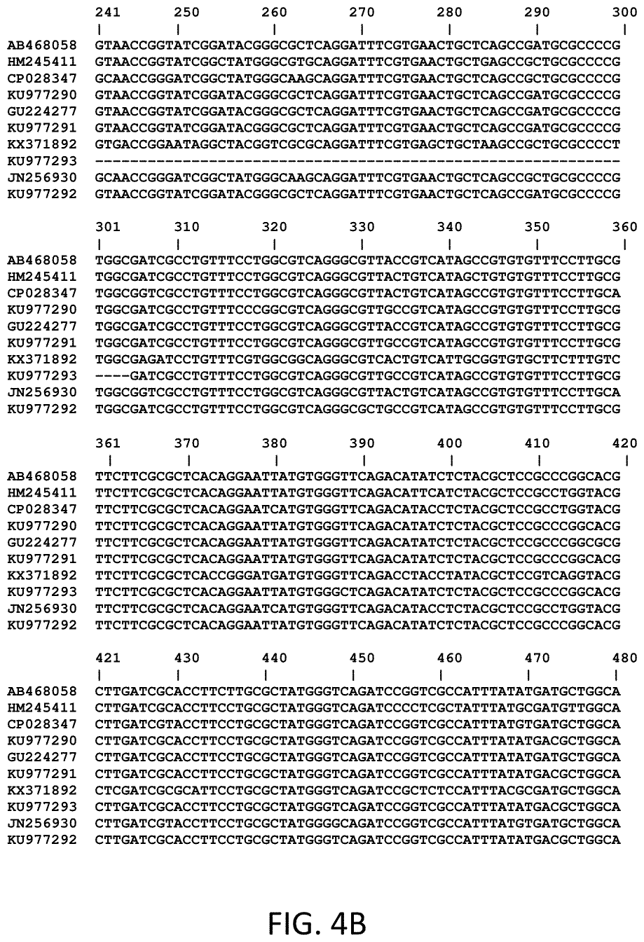

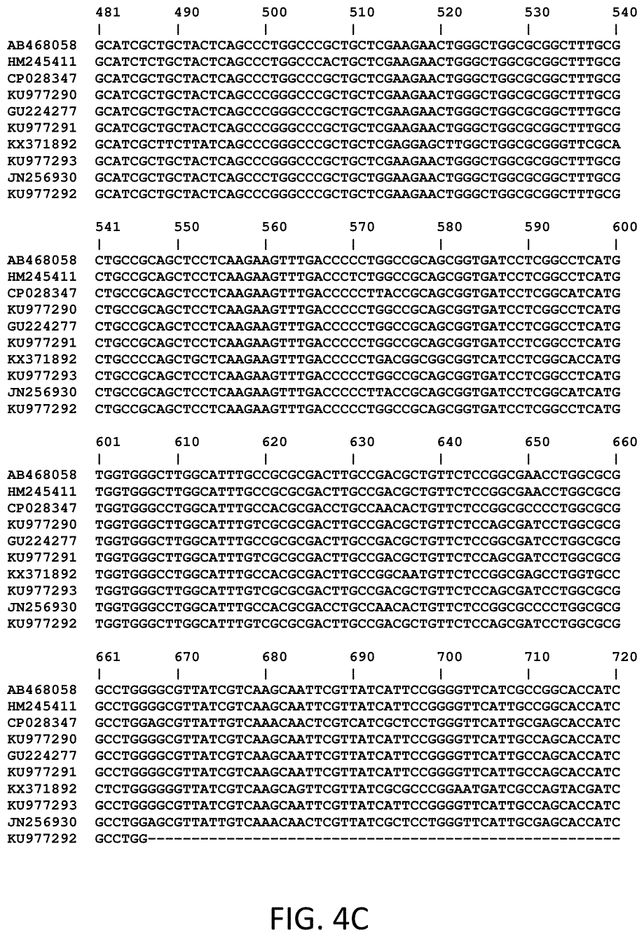

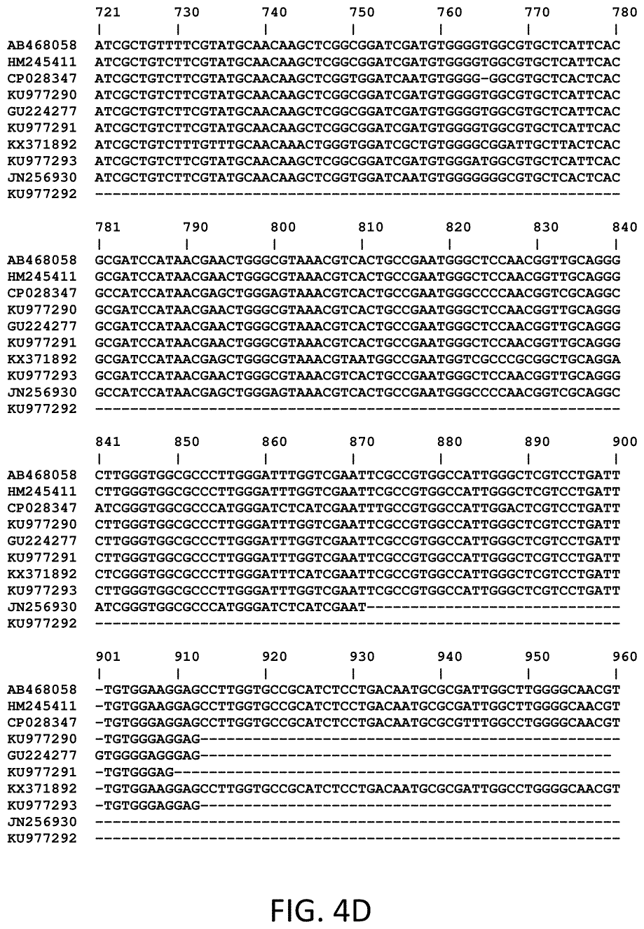

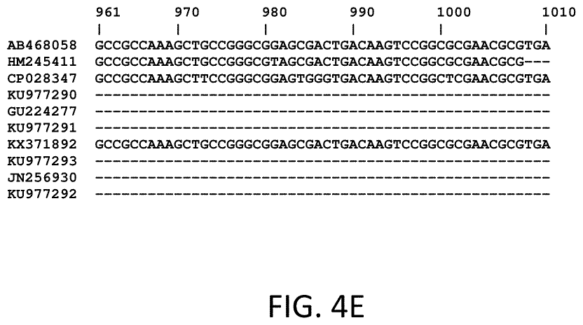

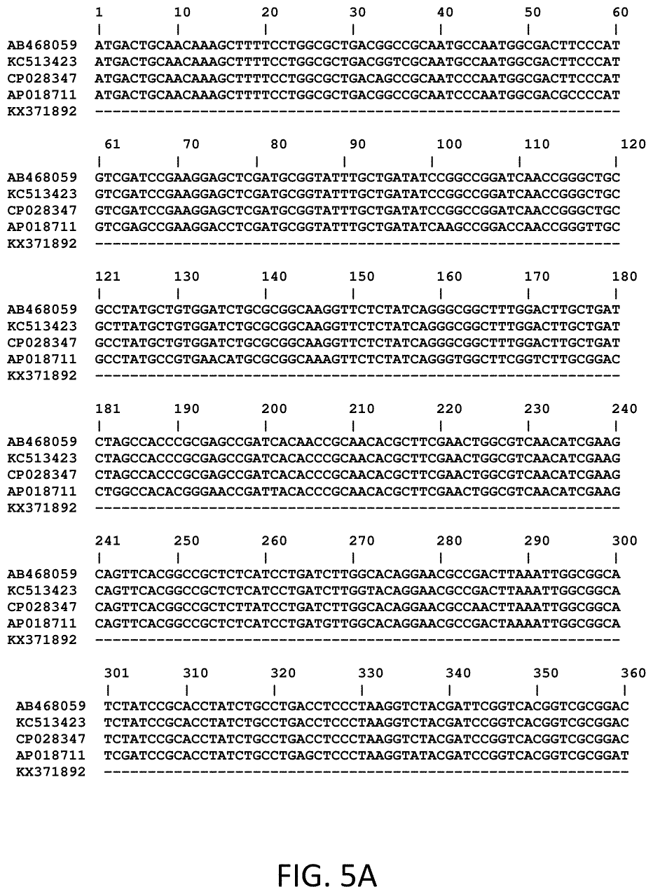

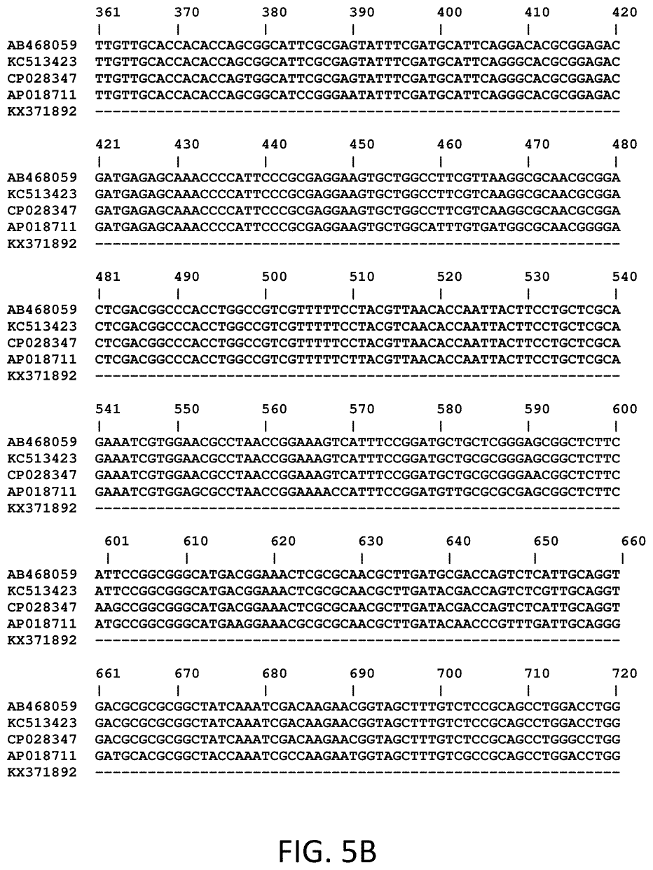

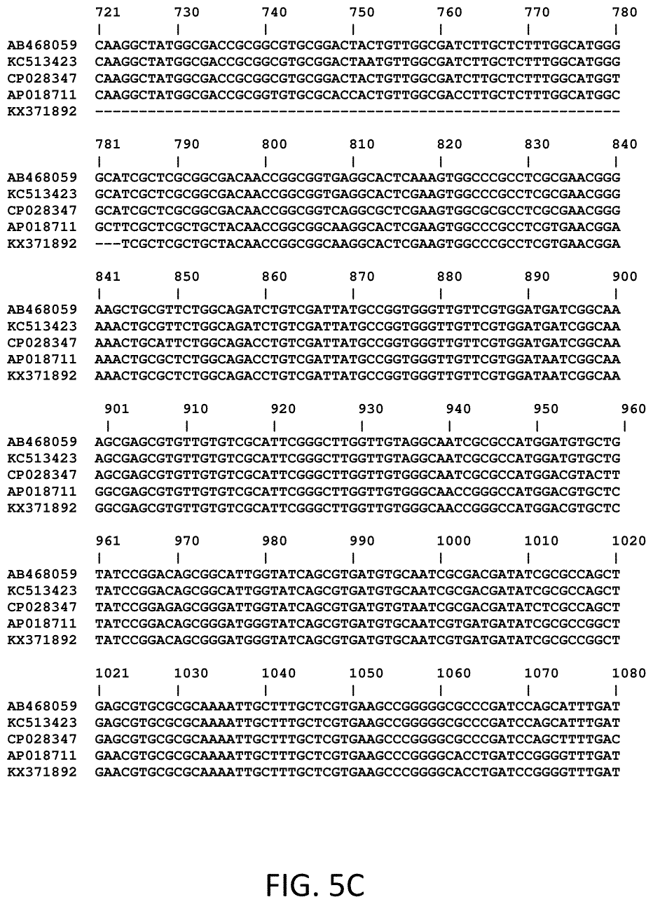

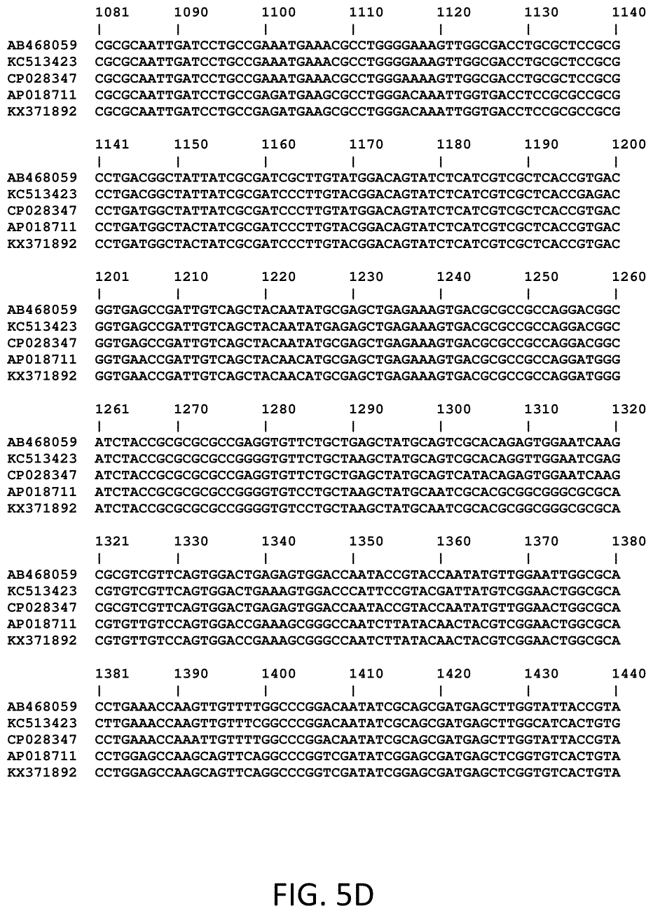

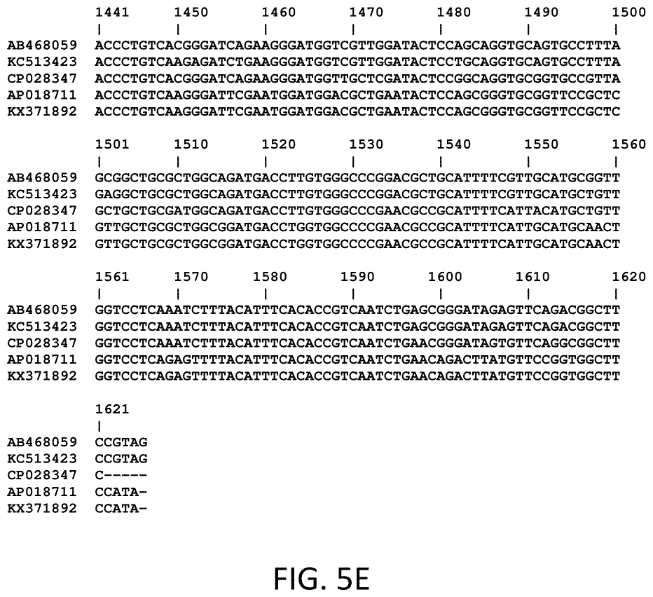

According to various embodiments, the recombinant protein has enzymatic activity of at least one of microcystinase and linearized microcystinase. As illustrated in FIG. 3, the MlrA microcystinase acts on cyclic MS specifically at the peptide bond between the 5th position amino acid (Adda) and the variable 4th position amino acid (Arg in MC-LR), which opens the cyclic structure and linearizes the heptapeptide. The linearized MC is significantly less toxic than the native cyclic form. In the case of MC-LR, the linearized MC is about 160 times less toxic than the cyclic form.

The peptidase, or linearized microcystinase, of MlrB acts on a peptide bond of the linear MC, cleaving the linear heptapeptide into smaller peptides, facilitating complete degradation of the MC, and reducing the toxicity of MC even further.

According to various embodiments, the recombinant protein is modified to include additional amino acids at the N-terminus and/or C-terminus of the protein. In some embodiments, the additional amino acids provide an affinity tag, which interacts with a specific material, thus binding the recombinant protein to this material. Contaminants or by-products can then be readily removed by washing steps. Although not a complete list, embodiments of N-terminus and/or C-terminus modification include: His-tag addition, FLAG tag addition, GST fusion protein generation, MBP fusion protein generation, and CBM addition. According to an embodiment, the recombinant protein includes a 6×His-tag at the N-terminus of the protein.

In some embodiments, an amino acid spacer is included between the affinity tag and the recombinant protein. In various embodiments, the spacer is not more than 20, not more than 10, or not more than 5 amino acids in length. In some embodiments, the spacer contains the recognition sequence of a specific protease to be able to split off the affinity tag and the spacer or parts of the spacer from the recombinant protein.

According to various embodiments, a purified recombinant protein is useful for applications in which the MlrA enzyme and/or MlrB enzyme are generally utilized. For example, the purified protein(s) can be made into, or incorporated into, a final product that is either liquid (solution, slurry) or solid (granular, powder). In some embodiments, the recombinant protein is partially purified, and for some applications the recombinant protein may not require further purification. For example, whole broth host cell culture can be used without further treatment or can be processed into a granule or microgranules.

A second aspect of the present specification relates to a composition that includes at least one recombinant protein having enzyme activity against MC. The at least one recombinant protein includes MlrA, MlrB, or a combination of both MlrA and MlrB, and according to various embodiments, the at least one recombinant protein has at least MlrA enzyme activity, MlrB enzyme activity, or a combination of MlrA and MlrB enzyme activity against MC. In some embodiments, the at least one recombinant protein is produced by the recombinant cells and methods disclosed herein.

Various embodiments of the composition are in a dry form or a liquid form. Embodiments of the composition are shelf-stable. Dry form embodiments of the composition include lyophilized and freeze-dried forms of MlrA, MlrB, or combinations of MlrA and MlrB. Various embodiments of the composition include stabilizing agents, such as polyols, sugars, or sugar alcohols. In some embodiments, a dry form of the composition is reconstituted in water or other suitable liquid to produce the composition in a liquid form. Some embodiments of the composition include purified recombinant protein, and some embodiments of the composition include partially purified recombinant protein.

A third aspect of the present specification relates to recombinant nucleic acid molecules that encode one or more recombinant protein having enzyme activity against MC. The recombinant protein includes MlrA, MlrB, or a combination of both MlrA and MlrB, and according to various embodiments, the protein has at least MlrA enzyme activity, MlrB enzyme activity, or a combination of MlrA and MlrB enzyme activity against MC.

According to various embodiments, the recombinant nucleic acid encodes for MlrA protein having the amino acid sequence of SEQ ID NO: 5, or having at least 70% identity to the amino acid sequence of SEQ ID NO: 5 and having enzymatic activity against cyclic MC. In various embodiments, the recombinant nucleic acid molecule encodes for MlrA protein having at least 75%, 80%, 85%, 90%, 95%, or 98% identity to the amino acid sequence of SEQ ID NO: 5 and having enzymatic activity against cyclic MC.

According to various embodiment, the recombinant nucleic acid encoding for MlrA protein has the nucleic acid sequence of SEQ ID NO: 1 or SEQ ID NO: 2, or has at least 70% identity to the nucleic acid sequence of SEQ ID NO: 1 or SEQ ID NO: 2. In various embodiments the recombinant nucleic acid has at least 75%, 80%, 85%, 90%, 95%, or 98% identity to the nucleic acid sequence of SEQ ID NO: 1 or SEQ ID NO: 2.

According to various embodiments, the recombinant nucleic acid encoding for MlrA protein has the nucleic acid sequence selected from SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or SEQ ID NO: 26, or has at least 70% identity to the nucleic acid sequence of SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or SEQ ID NO: 26. In various embodiments the recombinant nucleic acid has at least 75%, 80%, 85%, 90%, 95%, or 98% identity to the nucleic acid sequence of SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or SEQ ID NO: 26.

According to various embodiments, the recombinant nucleic acid encodes for MlrB protein having the amino acid sequence of SEQ ID NO: 6, or having at least 70% identity to the amino acid sequence of SEQ ID NO: 6 and having enzymatic activity against linearized MC. In various embodiments, the recombinant nucleic acid molecule encodes for MlrB protein having at least 75%, 80%, 85%, 90%, 95%, or 98% identity to the amino acid sequence of SEQ ID NO: 6 and having enzymatic activity against linearized MC.

According to various embodiment, the recombinant nucleic acid encoding for MlrB protein has the nucleic acid sequence of SEQ ID NO: 3 or SEQ ID NO: 4, or has at least 70% identity to the nucleic acid sequence of SEQ ID NO: 3 or SEQ ID NO: 4. In various embodiments, the recombinant nucleic acid has at least 75%, 80%, 85%, 90%, 95%, or 98% identity to the nucleic acid sequence of SEQ ID NO: 3 or SEQ ID NO: 4.

According to various embodiment, the recombinant nucleic acid encoding for MlrB protein has the nucleic acid sequence of SEQ ID NO: 28, SEQ ID NO: 30, SEQ ID NO: 32, or SEQ ID NO: 34, or has at least 70% identity to the nucleic acid sequence of SEQ ID NO: 28, SEQ ID NO: 30, SEQ ID NO: 32, or SEQ ID NO: 34. In various embodiments, the recombinant nucleic acid has at least 75%, 80%, 85%, 90%, 95%, or 98% identity to the nucleic acid sequence of SEQ ID NO: 28, SEQ ID NO: 30, SEQ ID NO: 32, or SEQ ID NO: 34.

According to various embodiments, the recombinant nucleic acid further encodes additional amino acids at the N-terminus and/or C-terminus of the protein. In some embodiments, the additional amino acids are an affinity tag. In an embodiment, the nucleic acid sequence that encodes the affinity tag is attached to the 3′ end of the sequence that encodes the recombinant protein, so that the affinity tag is fused to the C-terminus of the protein. In another embodiment, the nucleic acid sequence that encodes the affinity tag is attached to the 5′ end of the sequence that encodes the recombinant protein, so that the affinity tag is fused to the N-terminus of the protein. Although not a complete list, embodiments of the additional amino acids at the N-terminus and/or C-terminus include: His-tag addition, FLAG tag addition, GST fusion protein generation, MBP fusion protein generation, and CBM addition. According to an embodiment, the recombinant protein includes a 6×His-tag at the C-terminus of the protein.

According to various embodiments, the recombinant nucleic acid is contained within a recombinant vector, such as a plasmid vector. Various embodiments include an expression vector containing the recombinant nucleic acid operably linked to one or more promoter elements that provide for expression of the recombinant protein in prokaryotic or eukaryotic cells. The promoter element is not particularly limited, as long as it can be expressed in the host cell, and several promoter sequences that are functional in prokaryotic and eukaryotic cells are known in the art. According to various embodiments, expression of the recombinant protein is controlled by a constitutive or inducible promoter. While constitutive promoters are active in all circumstances, inducible promoters are active in the cell only in response to specific stimuli, such as the presence of an external factor. Although not limited, in some embodiments the vector is pET-21a (Genscript, Piscataway, N.J.) and the recombinant nucleic acid is operably linked to the T7 promoter. In some embodiments, the vector contains a lac operon in which expression of the recombinant protein is controlled by the presence or absence of isopropyl β-D-1-thiogalactopyranoside (IPTG).

According to various embodiments, the recombinant nucleic acid is contained within a recombinant vector, and the nucleic acid includes a first gene mlrA encoding a recombinant MlrA protein and a second gene mlrB encoding a recombinant MlrB protein. In some embodiments of the nucleic acid, the mlrA and mlrB genes have a bicistronic arrangement in the recombinant vector.

Another aspect of the present specification relates to recombinant cells that contain a heterologous gene encoding a recombinant protein having enzymatic activity against MC. The heterologous gene includes a recombinant nucleic acid molecule as disclosed herein. According to various embodiments, the heterologous gene is at least one of mlrA and mlrB, or a combination of both mlrA and mlrB, and the recombinant protein includes MlrA, MlrB, or a combination of both MlrA and MlrB. According to various embodiments, the recombinant protein has MlrA enzyme activity, MlrB enzyme activity, or a combination of MlrA and MlrB enzyme activity against MC.

In various embodiments of the recombinant cell, the heterologous gene encodes recombinant MlrA and MlrB proteins, and the coding sequences for MlrA and MlrB are in a bicistronic arrangement.

Various embodiments of the recombinant cells contain a recombinant vector as disclosed herein. According to various embodiments, the recombinant cells contain a recombinant nucleic acid contained within a recombinant vector, and the nucleic acid includes a first mlrA gene encoding a recombinant MlrA protein and a second gene mlrB encoding a recombinant MlrB protein. In some embodiments, the mlrA and mlrB genes have a bicistronic arrangement in the nucleic acid.

Embodiments of the recombinant cells expressing the recombinant protein having enzyme activity against MC are used to produce the recombinant protein by culturing the cells under conditions that permit expression of the protein. In some embodiments, the recombinant cell is prokaryotic, and in some embodiments, the recombinant cell is eukaryotic. According to various embodiments, the recombinant cell is a bacterial cell, a fungal cell, a yeast cell, an insect cell, a plant cell, or a mammalian cell. In some embodiments, the cell is a bacterial cell such as an Escherichia (e.g., E. coli), Bacillus (e.g., B. subtilis), Pseudomonas (e.g., P. putida), or Rhizobium (e.g., R. meliloti) cell, and in some embodiments, the cell is a yeast cell such as Saccharomyces cerevisiae, Schizosaccharomyces pombe, or Pichia pastoris.

According to various embodiments, the gene encoding the recombinant protein has been modified by codon optimization for expression in the recombinant cell. In some embodiments, the host cell is E. coli and the gene encoding the recombinant protein has been codon optimized for expression in E. coli.

According to various embodiments, the heterologous gene encoding the recombinant protein is at least one of mlrA and mlrB from a microorganism selected from the group consisting of Sphingopyxis, Sphingomona, Novosphingobium, Sphingosinicella, Stenotrophomonas, Catellibacterium, Kurthia, Rhizobium, Phyllobacterium, Actinoplanes, Pseudoxanthomonas, and Bacillus. In some embodiments, at least one of the mlrA and mlrB is from Sphingopyxis sp. C-1. In some embodiments, both mlrA and mlrB are from Sphingopyxis sp. C-1.

Another aspect of the present specification is directed to methods of producing recombinant MlrA and MlrB proteins having enzymatic activity against MC. The methods include culturing a recombinant host cell according to the disclosure herein. Various embodiments of the methods include culturing a recombinant host cell containing a heterologous gene encoding one or more recombinant protein having enzyme activity against MC to produce the protein. In some embodiments, the method also includes further steps for recovering and/or purifying the one or more protein from the cells. The one or more recombinant protein includes MlrA, MlrB, or a combination of both MlrA and MlrB, and according to various embodiments, the recombinant protein has MlrA enzyme activity, MlrB enzyme activity, or a combination of MlrA and MlrB enzyme activity against MC.

In some embodiments the host cell is prokaryotic and in some embodiments the host cell is eukaryotic. In various embodiments, the host cell is a bacterial cell, a fungal cell, a yeast cell, an insect cell, a plant cell, or a mammalian cell. Examples of host cells include bacteria Escherichia (e.g., E. coli), Bacillus (e.g., B. subtilis), Pseudomonas (e.g., P. putida), and Rhizobium (e.g., R. meliloti), and yeasts such as Saccharomyces cerevisiae, Schizosaccharomyces pombe, and Pichia pastoris. Conventionally known strains of E. coli, such as BL21 (DE3), K12, DH1, or JM 109 strains can be used, and B. subtilis 168 strain or the like can be used.

According to various embodiments, the gene encoding the recombinant protein has been modified by codon optimization for expression in the recombinant host cell. In some embodiments, the host cell is E. coli and the gene encoding the recombinant protein has been codon optimized for expression in E. coll.

According to various embodiments of the method of producing recombinant proteins having enzyme activity against MC, the recombinant host cells are cultivated using a fed-batch protocol. In this case, fed-batch is understood to mean that a portion of the nutrients is already present at the beginning of the cultivation and a further portion of the nutrients is added continuously or discontinuously from a specific point in time. In other embodiments, the host cells are cultivated using a batch protocol. In this case, batch is understood to mean that all the nutrients are already present at the beginning of cultivation and no further nutrients are added during cultivation.

For large-scale or industrial-scale production of recombinant proteins, in various embodiments, the recombinant host cells are cultured in fermenters that are adapted accordingly to the metabolic properties of the cells. During the culture, the host cells metabolize the supplied substrate and form the desired product (i.e., recombinant protein), which after the end of fermentation, in some embodiments, is separated from the production cells and is purified and/or concentrated from the fermenter slurry and/or the fermentation medium. In some embodiments, methods for producing the recombinant protein do not include a purification step that serves for the targeted separation of the protein. Also, some embodiments include recombinant protein preparations obtainable by the present method that does not include a purification step for the targeted separation of the protein.

Another aspect of the present specification is directed to methods of degrading MC. Degrading MC includes cleaving one or more peptide bonds in a native circular MC, in a linear MC, and/or in a product of such peptide bond cleavage, such as the tetrapeptide exemplified in FIG. 3. In some embodiments, degrading MC includes one or more of the enzyme activities of MlrA and MlrB, and one or more of the microcystinase, linear-microcystinase, and tetrapeptidase activities illustrated in FIG. 3. In embodiments of the method, degrading the MC significantly reduces its toxicity.

In various embodiments, a method of degrading MC includes contacting the MC with a recombinant protein having enzymatic activity against MC. The recombinant protein includes MlrA, MlrB, or a combination both MlrA and MlrB, and according to various embodiments, the protein has MlrA enzyme activity, MlrB enzyme activity, or a combination of MlrA and MlrB enzyme activity against MC. The MlrA and MlrB enzymes utilize MC as a substrate and their enzyme activity, both individually and collectively, degrade and detoxify MC. According to various embodiments of the method, the enzymatic activity includes at least one of microcystinase and linear microcystinase.

Another aspect of the present specification is directed to methods of treating water contaminated by a harmful cyanobacterial/algal bloom. The contaminated water contains MC, and embodiments of the methods are useful for reducing the level of MC and/or for detoxifying the MC in the MC-contaminated water. The methods include a step of bringing the MC-contaminated water into contact with an effective amount of a recombinant protein having enzyme activity against MC. The recombinant protein includes MlrA, MlrB, or a combination of both MlrA and MlrB, and according to various embodiments, the protein has MlrA enzyme activity, MlrB enzyme activity, or a combination of MlrA and MlrB enzyme activity against MC.

In various embodiments of treating MC-contaminated water, the water is lake water, reservoir water, pond water, river water, or irrigation water. In some embodiments, the MC-contaminated water is water that serves as a source of drinking water and has become contaminated with unsafe levels of MC. In some embodiments, the method is used to treat MC-contaminated water before the water enters a standard water treatment process.

Levels of microcystins in the water to be treated can vary widely, for example from about 0.5 μg/L to about 1 g/L. Exemplary microcystin concentrations can be about 0.5 μg/L, about 1.0 μg/L, about 5.0 μg/L, about 10.0 μg/L, about 20.0 μg/L, about 50.0 μg/L, about 100 μg/L, about 200 μg/L, about 500 μg/L, about 1.0 mg/L, about 2.0 mg/L, about 5.0 mg/L, about 10.0 mg/L, about 20.0 mg/L, about 50.0 mg/L, about 100 mg/L, about 200 mg/L. about 500 mg/L or about 1 g/L.

Another aspect of the present specification is directed to a biofilter that includes a medium and a biofilm on the medium, wherein the biofilm is formed from a group of cells that includes the recombinant cells disclosed herein. In various embodiments, the recombinant cells contain a heterologous gene encoding a recombinant protein having enzymatic activity against MC, wherein the recombinant cells are capable of degrading MC. In embodiments of the biofilter, the medium develops a biological film (biofilm) of cells which feed on the MC in the water being filtered by the biofilter. The medium is any suitable medium which allows the cells to attach, grow, and develop into a well-established biofilm. In certain embodiments, the medium is one or more of sand, gravel, polyurethan foam, peat, compost, woodchips, seashells, plastics, pumice, siliconized glass, or any combination thereof.

Another aspect of the present specification is directed to a method of filtering water to remove MC by passing the water through a biofilter described herein. In various embodiments, the biofilter includes a medium and a biofilm on the medium, wherein the biofilm is formed from a group of cells that includes the recombinant cells disclosed herein. In various embodiments, the recombinant cells contain a heterologous gene encoding a recombinant protein having enzymatic activity against MC, wherein the recombinant cells are capable of degrading MC. In various embodiments, the water being filtered is lake water, reservoir water, pond water, river water, or irrigation water. In some embodiments, the water being filtered is drinking water.

### EXAMPLES

### Production of MlrA, MlrB, and MlrAB Producing Bacterial Strains

The mlrABCD gene cluster of Sphingopyxis sp. C-1 is encoded on an approximately 8.5 Kb section of the genome (FIG. 2), and the nucleic acid sequences of mlrA (SEQ ID NO: 1) and mlrB (SEQ ID NO: 3) genes are available in the NCBI database (Genbank accession #AB468058 and AB468059).

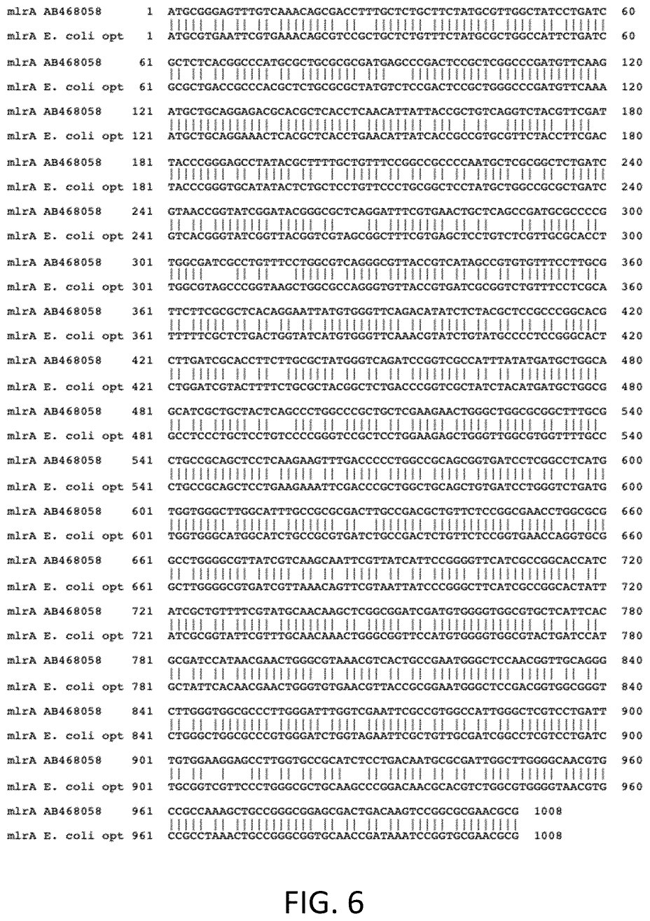

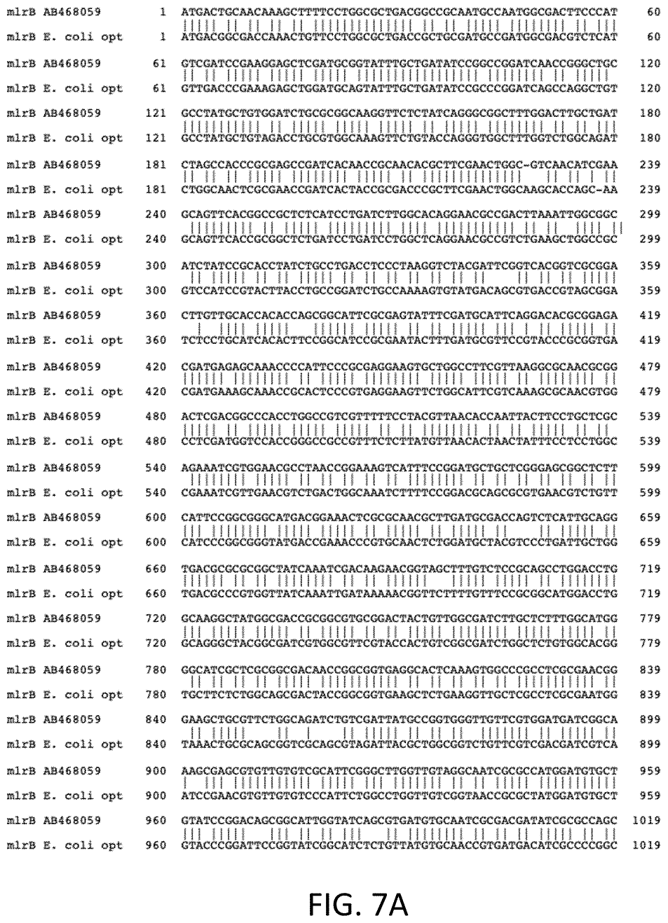

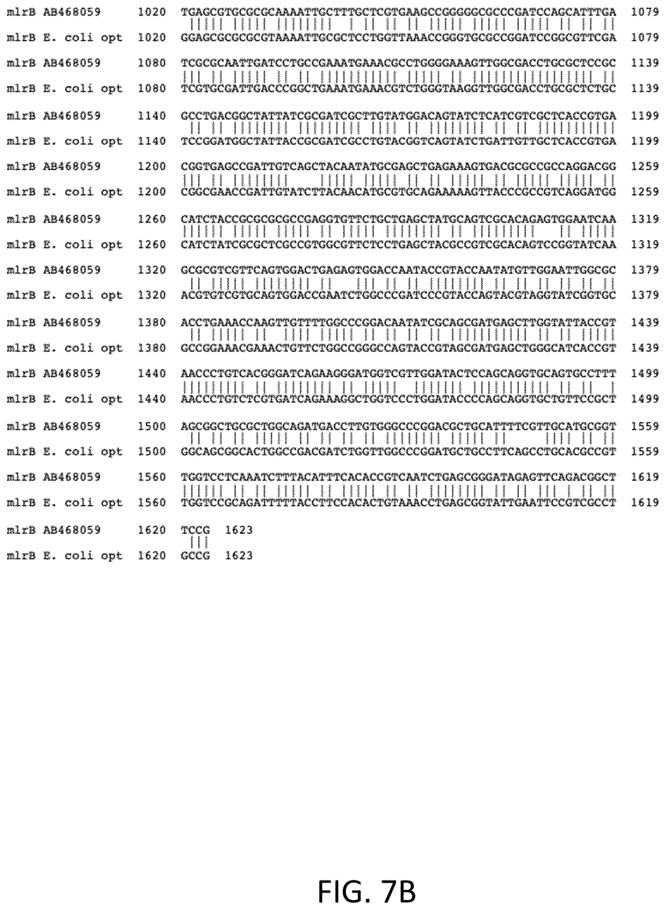

From these sequences, the mlrA and mlrB genes were codon optimized for expression in E. coli using Geneious Prime software, v2019.0.4 (Biomatters, Inc., San Diego, Calif.) (FIG. 6). From the codon optimized sequences, individual optimized mlrA (SEQ ID NO: 2) and optimized mlrB (SEQ ID NO: 4) nucleic acid sequences were chemically synthesized (Genscript, Piscataway, N.J.).

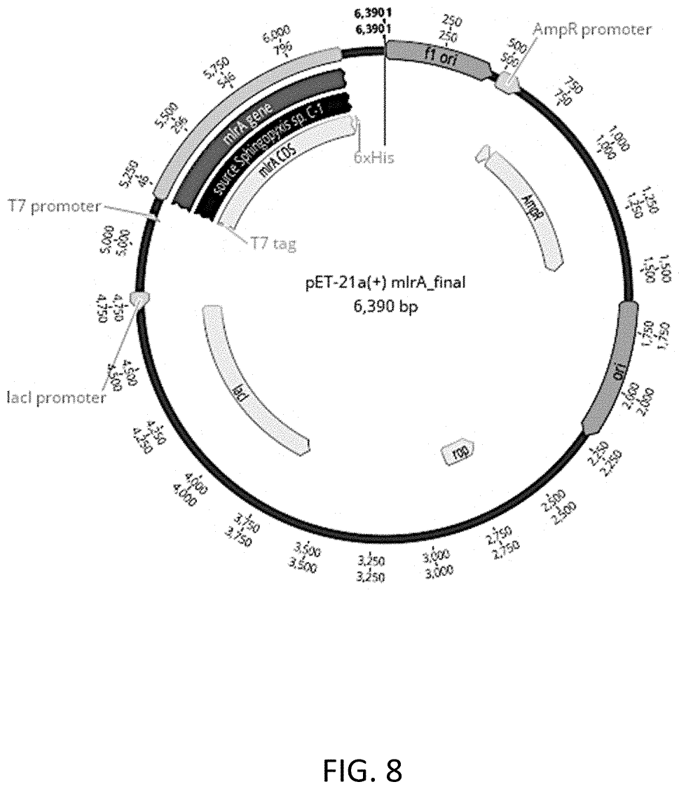

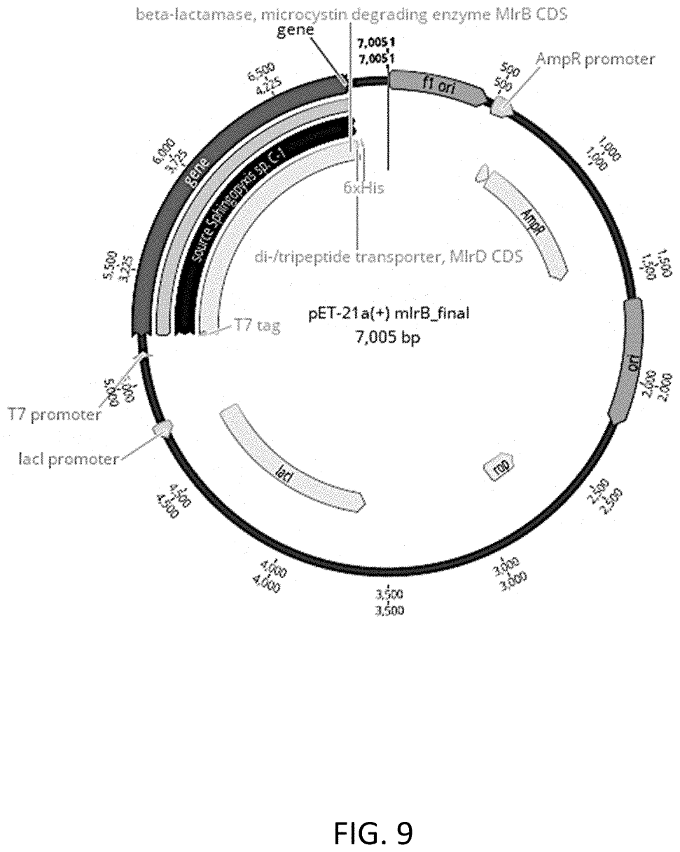

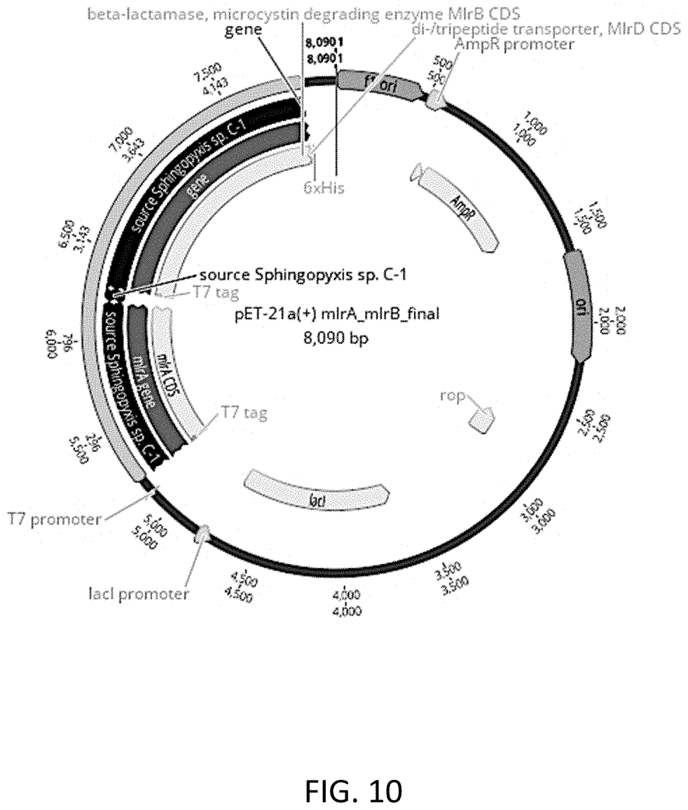

The optimized mlrA and mlrB nucleic acid sequences were individually subcloned into the pET21a expression vector (Genscript, Piscataway, N.J.), resulting in pET21a_mlrA (SEQ ID NO: 7) (FIG. 8) and pET21a_mlrB (SEQ ID NO: 8) (FIG. 9). The optimized mlrB nucleic acid was also subcloned into pET-21a_mlrA to produce a bicistronic mlrAB construct containing both mlrA and mlrB genes, pET21a_mlrA_mlrB (SEQ ID NO: 9) (FIG. 10). In this bicistronic mlrAB construct, the coding regions of mlrA and mlrB are in tandem and the construct retains a native intergenic noncoding region that follows mlrA and precedes mlrD in the native configuration of the mlrABCD locus. This construct is expected to produce stoichiometric amounts of MlrA and MlrB from the translation of a single bicistronic mRNA.

Each of the resulting vectors, pET21a_mlrA, pET21a_mlrB, and pET21a_mlrA_mlr_B was transformed into competent E. coli (BL21 (DE3)).

### LC-MS Analysis of Microcystin

Liquid Chromatography-Mass Spectrometry (LC-MS) was used to quantify microcystin concentration. The analysis involved a direct injection method using an Agilent 1260 Infinity II LC-MS (Agilent Technologies, Santa Clara, Calif.) and a Zorbax SB-18 5 μm, 4.6×150 mm column (Agilent Technologies) with a flow rate of 1 ml/min and a 25 μl injection using a gradient of MeOH:0.1% Formic Acid starting at a ratio of 10:90 for 2 min, then 80:20 to 13 min, followed by 90:10 to 17 min, and back to 10:90 by 21 min and finishing at 24 min. The column oven was set at 30° C. and the variable wavelength detector was set at 238 nm. The MS was set in positive ion mode, with the SIM Ions at 995.5 m/z for the cyclic MC, 1013.7 m/z for the linear (AC) MC, and 615.3 m/z for the tetrapeptide (Dziga et al. 2012; Massey and Yang 2020). The fragmentor was set at 70, gain EMV at 1, and actual dwell at 590, the gas temperature was 300° C., drying gas was set at 8 L/min, and Neb pressure at 25 psig. Analytical grade microcystin-LR standard (Millipore Sigma, St. Louis, Mo.) diluted in water to varying concentrations was used to build standard curves.

### Assessing Growth and Enzyme Activity Against Microcystin

The E. coli strains containing mlrA, mlrB, mlrA+mlrB (as a combination of separate strains), and mlrAB (bicistronic mlrA and mlrB), were inoculated into 6 ml LB with 100 ppm carbenicillin, 1 mM IPTG, and 10 ppm MC. A sterile control was also included. A sample of the medium (1 ml) was immediately taken (0 h), filtered through a 0.45 μm syringe filter, and placed in an HPLC vial for direct injection into the HPLC. Another sample was taken after 24 h incubation of the cultures at 37° C., shaking at 100 rpm.

Growth of the strains in a minimal medium was assessed. Growth in the minimal medium (M9, 0.5% Glucose, 0.5% YE, 1 mM IPTG, 100 ppm Carbenicillin) was analyzed, and degradation of MC and the various breakdown products was assessed by LC-MS. Cultures were started at an OD₆₀₀˜0.05, in triplicate and both growth (OD₆₀₀) and MC degradation were monitored at time 0 h, 3 h, and 21 h.

### Cell-Free Media Filtrates

Crude, cell-free filtrates for each of the constructs were collected and monitored for enzyme activity via the degradation of microcystin. Cultures (30 ml in 50 ml Falcon tubes) from mlrA, mlrAB, and BL21 control strains were grown for 24 h in LB plus 100 ppm carbenicillin (not added to control), with and without 1 mM IPTG. The cultures were then centrifuged at 7,000×g for 10 min, 5 ml of supernatant was filtered with 0.45 μm PFTE filter, and 10 ppm MC was added to the supernatant. Aliquots of 0.5 ml were added to HPLC vials, left at room temperature, and monitored over 72 h by LC-MS.

Because the culture media contained presumptively dilute concentrations of the enzymes, a method to concentrate the enzymes was investigated. The mlrAB strain was grown overnight in 100 ml LB at 37° C. with shaking and cells were separated from the supernatant by centrifugation. The supernatant was then filtered consecutively through an AMICON® Ultra-15 Centrifugal Filter Unit (MilliporeSigma, Burlington, Mass.), and 10 kDa cellulose membrane cartridge following the manufacturer's instructions. After the supernatant had passed through, the cartridge was then rinsed with 5 ml TE (pH 7.5) and then 2 ml TE was added, vortexed and collected (50×concentration). A blank LB medium control was performed to account for background protein.

Bradford assays were performed with a commercially available Quick Start Bovine Serum Albumin Standard Set and Bradford Reagent (Bio-Rad, Hercules, Calif.) to quantify total protein according to the manufacturer's instructions. Enzyme activity was assessed by degradation of MC. Because this was just a crude separation, different concentrations of filtrate (% volume) were added to 10 ppm MC (5 ul) in water (Table 1) in an HPLC vial and allowed to incubate at room temperature. HPLC samples were taken at 24 h and 48 h.

TABLE 1
Crude filtrate setup volumes for assessment of enzyme activity.

| | | | 50% | 25% | 10% | 1% | 0.1% |
|---|---|---|---|---|---|---|---|
| | 0 MC | MC | Conc | Conc | Conc | Conc | Conc |
| water (ul) | 250 | 495 | 245 | 370 | 445 | 490 | 494.5 |
| MC 10 ppm (ul) | 0 | 5 | 5 | 5 | 5 | 5 | 5 |
| Filtrate (ul) | 250 | 0 | 250 | 125 | 50 | 5 | 0.5 |

### Isolation and Characterization of Purified Enzymes

The recombinant nucleic acid constructs were engineered to express N-terminal 6×His tags to further facilitate rapid purification of the MlrA, MlrB and MlrAB proteins from the recombinant E. coli strains. These enzymes can be concentrated first by collecting the supernatant through centrifugation and adding it to an AM ICON® 500 ml Stirred Cells filtration unit containing a BIOMAX® PES 30 kDa Ultrafiltration Membrane (MilliporeSigma, Burlington, Mass.). Proteins greater than 30 kDa can be collected on the membrane surface and then removed by physical agitation in saline buffer and purified via affinity chromatography using Ni-NTA columns (Qiagen, Germantown, Md.) which contain nickel that tightly binds to the 6×His tag.

The Ni-NTA purified enzymes can be further evaluated separately for the remediation of microcystins in various matrices and enzyme kinetics can be performed on each. Basic protein characterizations can be performed on MlrA, MlrB, and MlrAB to approximate the amount of protein trapped intracellularly versus that secreted outside the cell. Bradford assays can be performed to quantify total protein and protein size and relative purity can be assessed using SDS-PAGE.

MlrA, MlrB, and MlrAB enzyme activity against MC (spiked at different levels) in various matrices can be assessed. Kinetics of the enzymes can be determined (i.e., reaction rates, upper and lower concentrations of substrate interactions, how matrix interference affects these rates) through several experimental variables: Clean reagent water/buffer system; Clean tap water; Clean aquaculture water; District assistance; Creek/lake/river water with low turbidity not suspected to have HAB prevalence; Creek/lake/river with high turbidity not suspected to have HAB prevalence; Real world HAB affected water with active toxin present.

### RESULTS

### Assessing Enzyme Activity Against Microcystin

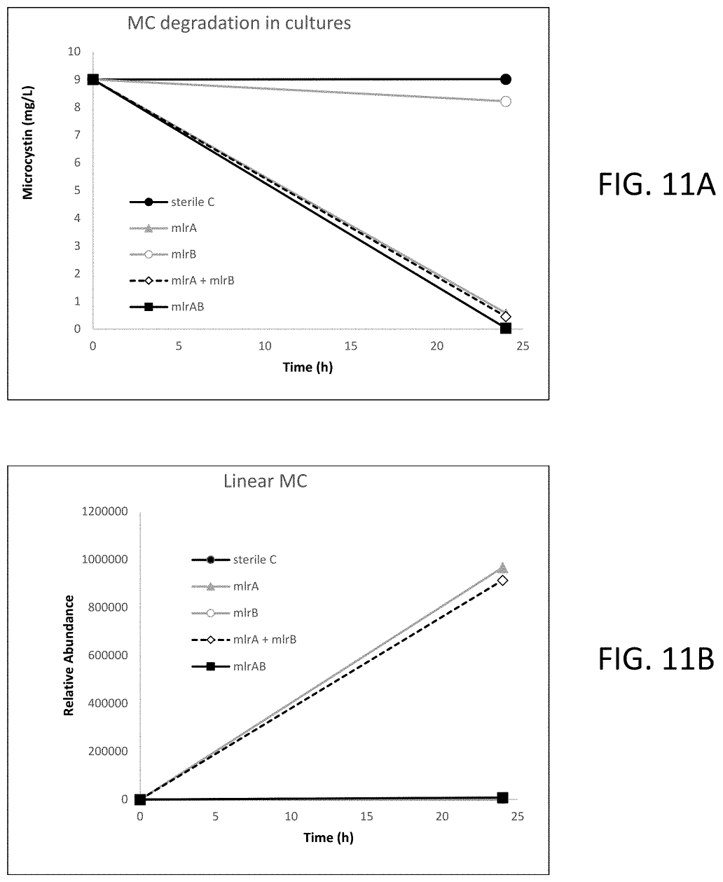

The degradation of native cyclic MC was assessed by LC-MS. The recombinant MlrA protein expressed in the E. coli mlrA strain almost completely degraded the cyclic MC in the culture medium after 24 h (FIG. 11A) and resulted in the accumulation of linear MC (FIG. 11B). As expected, the recombinant MlrB protein expressed in the culture medium of the mlrB strain was not active against the cyclic MC. The combination of MlrA+MlrB proteins from a combination of the separate culture mediums also degraded cyclic MC and resulted in the accumulation of linear MC, with little difference noted from that of MlrA protein alone. The mlrAB strain expressing both MlrA protein and MlrB protein in a bicistronic arrangement completely degraded the cyclic MC after 24 h (FIG. 11A) and further degraded the linear MC (FIG. 11B). The bicistronic arrangement presumably providing stoichiometric amounts of MlrA and MlrB completely degraded the cyclic MC (FIG. 11A) and linear MC (FIG. 11B), indicating exceptional MlrA and MlrB enzyme activity.

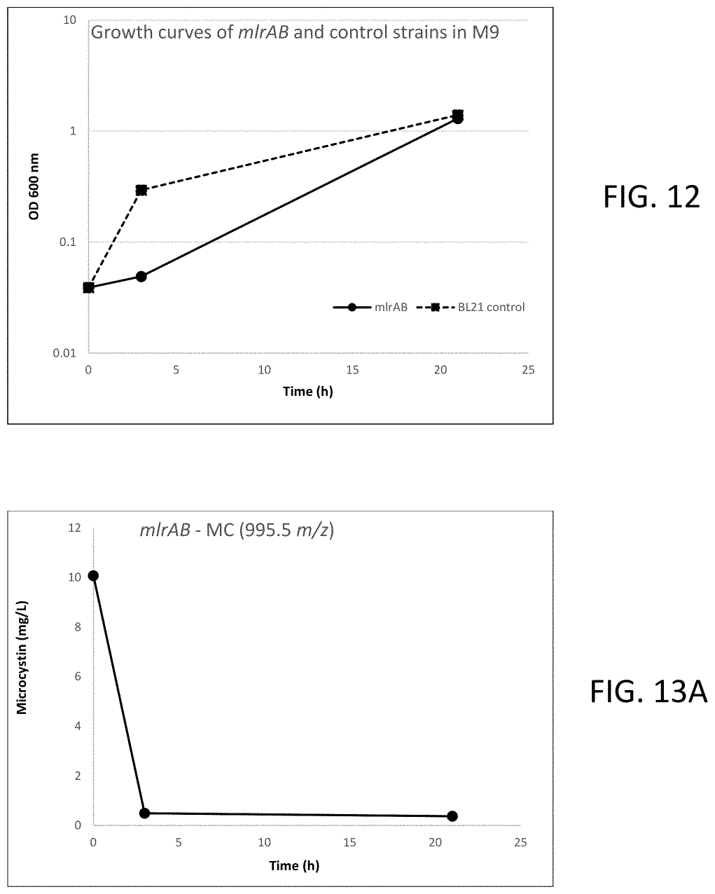

To aid in enzyme purification by minimizing growth medium background, the minimal medium M9 was used to grow the E. coli BL21 control strain and the mlrAB strain. The growth rate of the mlrAB strain initially appeared to be impacted and grew slower than the BL21 control. However, final cell OD in the cultures was similar, indicating that overall cell density was not impacted, but the initial growth phase was delayed in the mlrAB cells. (FIG. 12).

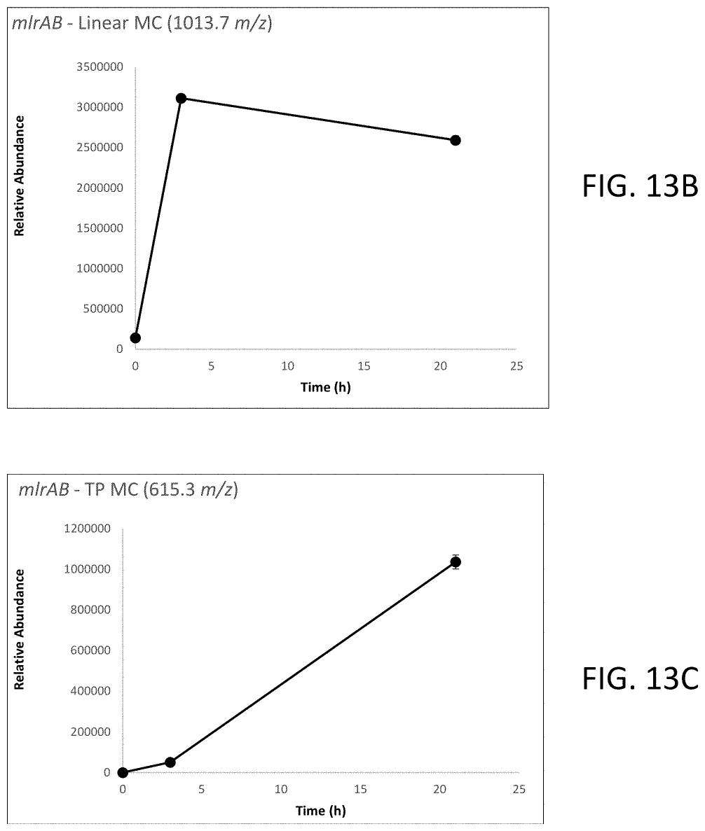

Regardless of the initially stymied growth of the mlrAB strain, the production and enzyme activity of the MlrA and MlrB proteins were active at the onset of cell growth as the MC was almost entirely degraded in the first 3 h (FIG. 13A). Concurrent with the degradation of the cyclic MC by MlrA protein was the emergence of the linear MC (FIG. 13B), followed by its degradation to the tetrapeptide structure (TP) by MlrB protein (FIG. 13C).

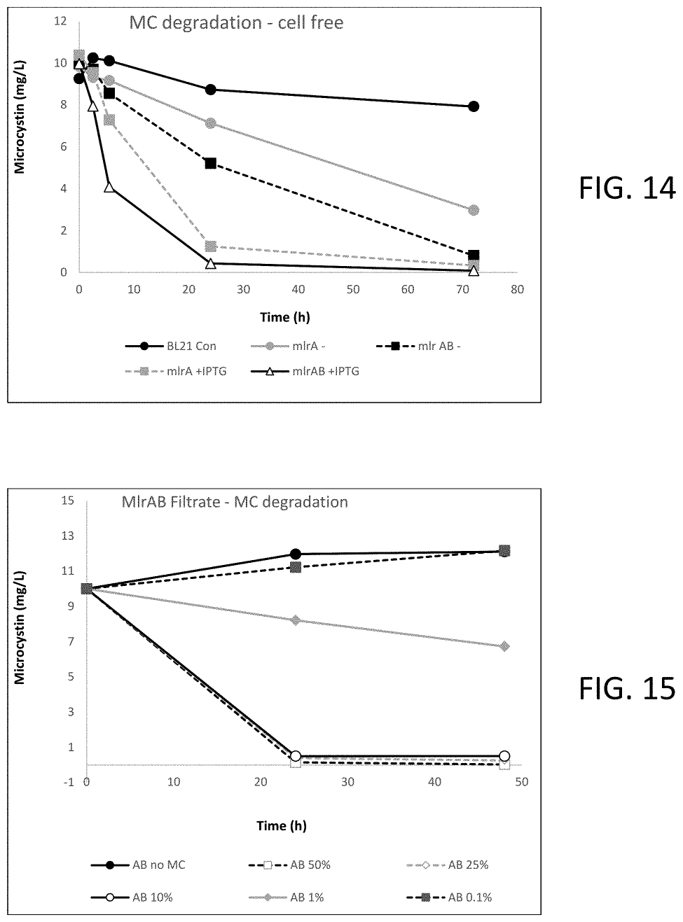

Cell free media collected from the mlrA and mlrAB strains grown with and without IPTG induction was analyzed for microcystin degradation activity. The cell free media contains active enzymes as the medium containing the mlrA strain and the medium containing the mlrAB strain degraded the MC. (FIG. 14). Expression of the mlr genes was “leaky” in both construct strains as MC degradation occurred in both constructs, albeit slower without the IPTG induction. Nevertheless, this indicated that the MlrA and MlrB proteins were being secreted out of the cells.

Further isolation of the MlrA and MlrB proteins by filtration with a 15 ml AMICON® filter and 50× concentration recovered 3.34 mg/ml, while 1.24 mg/ml protein was recovered from LB medium alone. Various concentrations (% by volume) of the filtrate were added to 10 mg/L MC and monitored at 24 h and 48 h for degradation of MC (FIG. 15). These data indicate that by 24 h all the microcystin was degraded in the 50% (850 μg), 25% (425 μg), and 10% (170 μg) filtrate groups, which equates to roughly 0.4 mg/L MC degraded/h. The 1% (17 μg) filtrate group degraded MC at a rate of 0.075 mg/L/h which is likely to be more accurate according to the slope and data points for this group. Using the 1% group data it can be inferred that 0.004 mg/L/h can be degraded by 1 μg of crude protein filtrate.

### SUMMARY

As expected, the mlrA strain linearized the cyclic MC to the predicted linear MC as detected by LC-MS, which accumulated as the end-product. The mlrAB strain also linearized the cyclic MC but the linear MC product did not accumulate and was quickly further broken down to the tetrapeptide by the predicted enzyme activity of MlrB protein. The tetrapeptide appears to be unstable and did not accumulate (data not shown) leading to even further breakdown of the MC toxin. A combination of MlrA and MlrB proteins from separate cultures of mlrA and mlrB strains yielded similar results to the bicistronic mlrAB strain.

Concentration of the MlrA and MlrB proteins in growth media through size exclusion by ultra-filtration reveals that the proteins are excreted from the bacterial cells and are active.

In view of the many possible embodiments to which the principles of the present disclosure may be applied, it should be recognized that the illustrated embodiments are only examples of the present disclosure and should not be taken as limiting the scope of this disclosure. Rather the scope of the present disclosure is defined in part by the following claims.

What is claimed is:

1. A combination of recombinant proteins, the combination comprising MlrA and MlrB, the combination of recombinant proteins having enzymatic activity against microcystin.

2. The combination of recombinant proteins according to claim 1, wherein at least one of the MlrA and MlrB is from a microorganism selected from the group consisting of: Sphingopyxis, Sphingomona, Novosphingobium, Sphingosinicella, Stenotrophomonas, Catellibacterium, Kurthia, Rhizobium, Phyllobacterium, Actinoplanes, Pseudoxanthomonas, and Bacillus.

3. The combination of recombinant proteins according to claim 1, wherein at least one of the MlrA and MlrB is from Sphingopyxis sp. C-1.

4. The combination of recombinant proteins according to claim 1, wherein:
the MlrA protein has the amino acid sequence of SEQ ID NO: 5, or has at least 70% identity to the amino acid sequence of SEQ ID NO: 5 and has enzymatic activity against cyclic microcystin; and
the MlrB protein has the amino acid sequence of SEQ ID NO: 6, or has at least 70% identity to the amino acid sequence of SEQ ID NO: 6 and has enzymatic activity against linearized microcystin.

5. The combination of recombinant proteins according to claim 1, wherein the microcystin is selected from the group consisting of Microcystin-LR, Microcystin-RR, Microcystin-YR, Microcystin-LA, Microcystin-LY, Microcystin-LW, and Microcystin-LF.

6. The combination of recombinant proteins according to claim 1, wherein the enzymatic activity is microcystinase and linearized microcystinase.

7. A composition comprising the combination of recombinant proteins according to claim 1, the composition having enzymatic activity against microcystin.

8. A recombinant nucleic acid encoding recombinant MlrA protein and MlrB protein, the recombinant MlrA protein and MlrB protein having enzymatic activity against microcystin.

9. The recombinant nucleic acid according to claim 8, wherein:
the nucleic acid encodes for MlrA protein having the amino acid sequence of SEQ ID NO: 5, or having at least 70% identity to the amino acid sequence of SEQ ID NO: 5 and having enzymatic activity against cyclic microcystin; and
the nucleic acid encodes for MlrB having the amino acid sequence of SEQ ID NO: 6, or having at least 70% identity to the amino acid sequence of SEQ ID NO: 6 and having enzymatic activity against linearized microcystin.

10. The recombinant nucleic acid according to claim 8, comprising a first gene encoding the recombinant MlrA protein and a second gene encoding the recombinant MlrB protein, wherein the first and second genes have a bicistronic arrangement in the nucleic acid.

11. A method of degrading microcystin, the method comprising contacting the microcystin with a combination of recombinant MlrA and MlrB proteins, the combination of recombinant MlrA and MlrB proteins having enzymatic activity against microcystin.

12. A method of reducing the level of microcystin in MC-containing water, the method comprising bringing the water into contact with a combination of recombinant MlrA and MlrB proteins, the combination of recombinant MlrA and MlrB proteins having enzymatic activity against microcystin.

13. The method according to claim 12, wherein the microcystin is contained in water contaminated by a harmful cyanobacterial/algal bloom.

14. The method according to claim 12, wherein the MC-containing water is lake water, reservoir water, pond water, river water, irrigation water, or a source of drinking water.

15. A recombinant cell, comprising a heterologous gene encoding recombinant MlrA and MlrB proteins, the recombinant MlrA and MlrB proteins having enzymatic activity against microcystin.

16. The recombinant cell according to claim 15, wherein the heterologous gene comprises coding sequences for MlrA and MlrB in a bicistronic arrangement.

17. A method of producing recombinant MlrA and MlrB proteins having enzymatic activity against microcystin, comprising culturing the recombinant cell according to claim 15 in a culture medium.

18. The method according to claim 15, wherein the heterologous gene has been codon optimized for expression of the recombinant MlrA and MlrB proteins in the recombinant cell.


SEQUENCE LISTING (35 sequences)

SEQ ID NO: 1 - DNA, 1008 bp, Sphingopyxis sp. C-1
  gene (1)..(1008): mlrA
atgcgggagtttgtcaaacagcgacctttgctctgcttctatgcgttggctatcctgatcgctctcacggcccatgcgctgcgcgcgatgagcccgactccgctcggcccgatgttcaagatgctgcaggagacgcacgctcacctcaacattattaccgctgtcaggtctacgttcgattacccgggagcctatacgcttttgctgtttccggccgccccaatgctcgcggctctgatcgtaaccggtatcggatacgggcgctcaggatttcgtgaactgctcagccgatgcgccccgtggcgatcgcctgtttcctggcgtcagggcgttaccgtcatagccgtgtgtttccttgcgttcttcgcgctcacaggaattatgtgggttcagacatatctctacgctccgcccggcacgcttgatcgcaccttcttgcgctatgggtcagatccggtcgccatttatatgatgctggcagcatcgctgctactcagccctggcccgctgctcgaagaactgggctggcgcggctttgcgctgccgcagctcctcaagaagtttgaccccctggccgcagcggtgatcctcggcctcatgtggtgggcttggcatttgccgcgcgacttgccgacgctgttctccggcgaacctggcgcggcctggggcgttatcgtcaagcaattcgttatcattccggggttcatcgccggcaccatcatcgctgttttcgtatgcaacaagctcggcggatcgatgtggggtggcgtgctcattcacgcgatccataacgaactgggcgtaaacgtcactgccgaatgggctccaacggttgcagggcttgggtggcgcccttgggatttggtcgaattcgccgtggccattgggctcgtcctgatttgtggaaggagccttggtgccgcatctcctgacaatgcgcgattggcttggggcaacgtgccgccaaagctgccgggcggagcgactgacaagtccggcgcgaacgcg

SEQ ID NO: 2 - DNA, 1008 bp, Artificial
  mlrA E. coli codon optimized
atgcgtgaattcgtgaaacagcgtccgctgctctgtttctatgcgctggccattctgatcgcgctgaccgcccacgctctgcgcgctatgtctccgactccgctgggcccgatgttcaaaatgctgcaggaaactcacgctcacctgaacattatcaccgccgtgcgttctaccttcgactacccgggtgcatatactctgctcctgttccctgcggctcctatgctggccgcgctgatcgtcacgggtatcggttacggtcgtagcggctttcgtgagctcctgtctcgttgcgcaccttggcgtagcccggtaagctggcgccagggtgttaccgtgatcgcggtctgtttcctcgcatttttcgctctgactggtatcatgtgggttcaaacgtatctgtatgcccctccgggcactctggatcgtacttttctgcgctacggctctgacccggtcgctatctacatgatgctggcggcctccctgctcctgtccccgggtccgctcctggaagagctgggttggcgtggttttgccctgccgcagctcctgaagaaattcgacccgctggctgcagctgtgatcctgggtctgatgtggtgggcatggcatctgccgcgtgatctgccgactctgttctccggtgaaccaggtgcggcttggggcgtgatcgttaaacagttcgtaattatcccgggcttcatcgccggcactattatcgcggtattcgtttgcaacaaactgggcggttccatgtggggtggcgtactgatccatgctattcacaacgaactgggtgtgaacgttaccgcggaatgggctccgacggtggcgggtctgggctggcgcccgtgggatctggtagaattcgctgttgcgatcggcctcgtcctgatctgcggtcgttccctgggcgctgcaagcccggacaacgcacgtctggcgtggggtaacgtgccgcctaaactgccgggcggtgcaaccgataaatccggtgcgaacgct

SEQ ID NO: 3 - DNA, 1623 bp, Sphingopyxix sp. C-1
  gene (1)..(1623): mlrB
atgactgcaacaaagcttttcctggcgctgacggccgcaatgccaatggcgacttcccatgtcgatccgaaggagctcgatgcggtatttgctgatatccggccggatcaaccgggctgcgcctatgctgtggatctgcgcggcaaggttctctatcagggcggctttggacttgctgatctagccacccgcgagccgatcacaaccgcaacacgcttcgaactggcgtcaacatcgaagcagttcacggccgctctcatcctgatcttggcacaggaacgccgacttaaattggcggcatctatccgcacctatctgcctgacctccctaaggtctacgattcggtcacggtcgcggacttgttgcaccacaccagcggcattcgcgagtatttcgatgcattcaggacacgcggagacgatgagagcaaaccccattcccgcgaggaagtgctggccttcgttaaggcgcaacgcggactcgacggcccacctggccgtcgtttttcctacgttaacaccaattacttcctgctcgcagaaatcgtggaacgcctaaccggaaagtcatttccggatgctgctcgggagcggctcttcattccggcgggcatgacggaaactcgcgcaacgcttgatgcgaccagtctcattgcaggtgacgcgcgcggctatcaaatcgacaagaacggtagctttgtctccgcagcctggacctggcaaggctatggcgaccgcggcgtgcggactactgttggcgatcttgctctttggcatggggcatcgctcgcggcgacaaccggcggtgaggcactcaaagtggcccgcctcgcgaacgggaagctgcgttctggcagatctgtcgattatgccggtgggttgttcgtggatgatcggcaaagcgagcgtgttgtgtcgcattcgggcttggttgtaggcaatcgcgccatggatgtgctgtatccggacagcggcattggtatcagcgtgatgtgcaatcgcgacgatatcgcgccagctgagcgtgcgcgcaaaattgctttgctcgtgaagccgggggcgcccgatccagcatttgatcgcgcaattgatcctgccgaaatgaaacgcctggggaaagttggcgacctgcgctccgcgcctgacggctattatcgcgatcgcttgtatggacagtatctcatcgtcgctcaccgtgacggtgagccgattgtcagctacaatatgcgagctgagaaagtgacgcgccgccaggacggcatctaccgcgcgcgccgaggtgttctgctgagctatgcagtcgcacagagtggaatcaagcgcgtcgttcagtggactgagagtggaccaataccgtaccaatatgttggaattggcgcacctgaaaccaagttgttttggcccggacaatatcgcagcgatgagcttggtattaccgtaaccctgtcacgggatcagaagggatggtcgttggatactccagcaggtgcagtgcctttagcggctgcgctggcagatgaccttgtgggcccggacgctgcattttcgttgcatgcggttggtcctcaaatctttacatttcacaccgtcaatctgagcgggatagagttcagacggcttccg

SEQ ID NO: 4 - DNA, 1623 bp, Artificial
  mlrB E. coli codon optimized
atgacggcgaccaaactgttcctggcgctgaccgctgcgatgccgatggcgacgtctcatgttgacccgaaagagctggatgcagtatttgctgatatccgcccggatcagccaggctgtgcctatgctgtagacctgcgtggcaaagttctgtaccagggtggctttggtctggcagatctggcaactcgcgaaccgatcactaccgcgacccgcttcgaactggcaagcaccagcaagcagttcaccgcggctctgatcctgatcctggctcaggaacgccgtctgaagctggccgcgtccatccgtacttacctgccggatctgccaaaagtgtatgacagcgtgaccgtagcggatctcctgcatcacacttccggcatccgcgaatactttgatgcgttccgtacccgcggtgacgatgaaagcaaaccgcactcccgtgaggaagttctggcattcgtcaaagcgcaacgtggcctcgatggtccaccgggccgccgtttctcttatgttaacactaactatttcctcctggccgaaatcgttgaacgtctgactggcaaatcttttccggacgcagcgcgtgaacgtctgttcatcccggcgggtatgaccgaaacccgtgcaactctggatgctacgtccctgattgctggtgacgcccgtggttatcaaattgataaaaacggttcttttgtttccgcggcatggacctggcagggctacggcgatcgtggcgttcgtaccactgtcggcgatctggctctgtggcacggtgcttctctggcagcgactaccggcggtgaagctctgaaggttgctcgcctcgcgaatggtaaactgcgcagcggtcgcagcgtagattacgctggcggtctgttcgtcgacgatcgtcaatccgaacgtgttgtgtcccattctggcctggttgtcggtaaccgcgctatggatgtgctgtacccggattccggtatcggcatctctgttatgtgcaaccgtgatgacatcgccccggcggagcgcgcgcgtaaaattgcgctcctggttaaaccgggtgcgccggatccggcgttcgatcgtgcgattgacccggctgaaatgaaacgtctgggtaaggttggcgacctgcgctctgctccggatggctattaccgcgatcgcctgtacggtcagtatctgattgttgctcaccgtgacggcgaaccgattgtatcttacaacatgcgtgcagaaaaagttacccgccgtcaggatggcatctatcgcgctcgccgtggcgttctcctgagctacgccgtcgcacagtccggtatcaaacgtgtcgtgcagtggaccgaatctggcccgatcccgtaccagtacgtaggtatcggtgcgccggaaacgaaactgttctggccgggccagtaccgtagcgatgagctgggcatcaccgtaaccctgtctcgtgatcagaaaggctggtccctggataccccagcaggtgctgttccgctggcagcggcactggccgacgatctggttggcccggatgctgccttcagcctgcacgccgttggtccgcagatttttaccttccacactgtaaacctgagcggtattgaattccgtcgcctgccg

SEQ ID NO: 5 - protein, 336 aa, Sphingopyxis sp. C-1
  MISC_FEATURE (1)..(336)
MREFVKQRPLLCFYALAILIALTAHALRAMSPTPLGPMFKMLQETHAHLNIITAVRSTFDYPGAYTLLLFPAAPMLAALIVTGIGYGRSGFRELLSRCAPWRSPVSWRQGVTVIAVCFLAFFALTGIMWVQTYLYAPPGTLDRTFLRYGSDPVAIYMMLAASLLLSPGPLLEELGWRGFALPQLLKKFDPLAAAVILGLMWWAWHLPRDLPTLFSGEPGAAWGVIVKQFVIIPGFIAGTIIAVFVCNKLGGSMWGGVLIHAIHNELGVNVTAEWAPTVAGLGWRPWDLVEFAVAIGLVLICGRSLGAASPDNARLAWGNVPPKLPGGATDKSGANA

SEQ ID NO: 6 - protein, 541 aa, Sphingpyxis sp. C-1
  MISC_FEATURE (1)..(541): mlrB
MTATKLFLALTAAMPMATSHVDPKELDAVFADIRPDQPGCAYAVDLRGKVLYQGGFGLADLATREPITTATRFELASTSKQFTAALILILAQERRLKLAASIRTYLPDLPKVYDSVTVADLLHHTSGIREYFDAFRTRGDDESKPHSREEVLAFVKAQRGLDGPPGRRFSYVNTNYFLLAEIVERLTGKSFPDAARERLFIPAGMTETRATLDATSLIAGDARGYQIDKNGSFVSAAWTWQGYGDRGVRTTVGDLALWHGASLAATTGGEALKVARLANGKLRSGRSVDYAGGLFVDDRQSERVVSHSGLVVGNRAMDVLYPDSGIGISVMCNRDDIAPAERARKIALLVKPGAPDPAFDRAIDPAEMKRLGKVGDLRSAPDGYYRDRLYGQYLIVAHRDGEPIVSYNMRAEKVTRRQDGIYRARRGVLLSYAVAQSGIKRVVQWTESGPIPYQYVGIGAPETKLFWPGQYRSDELGITVTLSRDQKGWSLDTPAGAVPLAAALADDLVGPDAAFSLHAVGPQIFTFHTVNLSGIEFRRLP

SEQ ID NO: 7 - DNA, 6390 bp, Artificial
  mlrA cod opt cloned in pET-21a
  gene (5205)..(6211): mlrA gene
  misc_feature (6234)..(6251): 6XHis tag
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcgtgaattcgtgaaacagcgtccgctgctctgtttctatgcgctggccattctgatcgcgctgaccgcccacgctctgcgcgctatgtctccgactccgctgggcccgatgttcaaaatgctgcaggaaactcacgctcacctgaacattatcaccgccgtgcgttctaccttcgactacccgggtgcatatactctgctcctgttccctgcggctcctatgctggccgcgctgatcgtcacgggtatcggttacggtcgtagcggctttcgtgagctcctgtctcgttgcgcaccttggcgtagcccggtaagctggcgccagggtgttaccgtgatcgcggtctgtttcctcgcatttttcgctctgactggtatcatgtgggttcaaacgtatctgtatgcccctccgggcactctggatcgtacttttctgcgctacggctctgacccggtcgctatctacatgatgctggcggcctccctgctcctgtccccgggtccgctcctggaagagctgggttggcgtggttttgccctgccgcagctcctgaagaaattcgacccgctggctgcagctgtgatcctgggtctgatgtggtgggcatggcatctgccgcgtgatctgccgactctgttctccggtgaaccaggtgcggcttggggcgtgatcgttaaacagttcgtaattatcccgggcttcatcgccggcactattatcgcggtattcgtttgcaacaaactgggcggttccatgtggggtggcgtactgatccatgctattcacaacgaactgggtgtgaacgttaccgcggaatgggctccgacggtggcgggtctgggctggcgcccgtgggatctggtagaattcgctgttgcgatcggcctcgtcctgatctgcggtcgttccctgggcgctgcaagcccggacaacgcacgtctggcgtggggtaacgtgccgcctaaactgccgggcggtgcaaccgataaatccggtgcgaacgctaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 8 - DNA, 7005 bp, Artificial
  mlrB cod opt cloned in pET-21a
  gene (5205)..(6827): mlrB gene
  misc_feature (6848)..(6866): 6XHis tag
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgacggcgaccaaactgttcctggcgctgaccgctgcgatgccgatggcgacgtctcatgttgacccgaaagagctggatgcagtatttgctgatatccgcccggatcagccaggctgtgcctatgctgtagacctgcgtggcaaagttctgtaccagggtggctttggtctggcagatctggcaactcgcgaaccgatcactaccgcgacccgcttcgaactggcaagcaccagcaagcagttcaccgcggctctgatcctgatcctggctcaggaacgccgtctgaagctggccgcgtccatccgtacttacctgccggatctgccaaaagtgtatgacagcgtgaccgtagcggatctcctgcatcacacttccggcatccgcgaatactttgatgcgttccgtacccgcggtgacgatgaaagcaaaccgcactcccgtgaggaagttctggcattcgtcaaagcgcaacgtggcctcgatggtccaccgggccgccgtttctcttatgttaacactaactatttcctcctggccgaaatcgttgaacgtctgactggcaaatcttttccggacgcagcgcgtgaacgtctgttcatcccggcgggtatgaccgaaacccgtgcaactctggatgctacgtccctgattgctggtgacgcccgtggttatcaaattgataaaaacggttcttttgtttccgcggcatggacctggcagggctacggcgatcgtggcgttcgtaccactgtcggcgatctggctctgtggcacggtgcttctctggcagcgactaccggcggtgaagctctgaaggttgctcgcctcgcgaatggtaaactgcgcagcggtcgcagcgtagattacgctggcggtctgttcgtcgacgatcgtcaatccgaacgtgttgtgtcccattctggcctggttgtcggtaaccgcgctatggatgtgctgtacccggattccggtatcggcatctctgttatgtgcaaccgtgatgacatcgccccggcggagcgcgcgcgtaaaattgcgctcctggttaaaccgggtgcgccggatccggcgttcgatcgtgcgattgacccggctgaaatgaaacgtctgggtaaggttggcgacctgcgctctgctccggatggctattaccgcgatcgcctgtacggtcagtatctgattgttgctcaccgtgacggcgaaccgattgtatcttacaacatgcgtgcagaaaaagttacccgccgtcaggatggcatctatcgcgctcgccgtggcgttctcctgagctacgccgtcgcacagtccggtatcaaacgtgtcgtgcagtggaccgaatctggcccgatcccgtaccagtacgtaggtatcggtgcgccggaaacgaaactgttctggccgggccagtaccgtagcgatgagctgggcatcaccgtaaccctgtctcgtgatcagaaaggctggtccctggataccccagcaggtgctgttccgctggcagcggcactggccgacgatctggttggcccggatgctgccttcagcctgcacgccgttggtccgcagatttttaccttccacactgtaaacctgagcggtattgaattccgtcgcctgccgaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 9 - DNA, 8090 bp, Artificial
  Bicistronic mlrA and mlrB cod opt cloned in pET-21a
  gene (5205)..(6212): mlrA gene
  gene (6287)..(7909): mlrB gene
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcgtgaattcgtgaaacagcgtccgctgctctgtttctatgcgctggccattctgatcgcgctgaccgcccacgctctgcgcgctatgtctccgactccgctgggcccgatgttcaaaatgctgcaggaaactcacgctcacctgaacattatcaccgccgtgcgttctaccttcgactacccgggtgcatatactctgctcctgttccctgcggctcctatgctggccgcgctgatcgtcacgggtatcggttacggtcgtagcggctttcgtgagctcctgtctcgttgcgcaccttggcgtagcccggtaagctggcgccagggtgttaccgtgatcgcggtctgtttcctcgcatttttcgctctgactggtatcatgtgggttcaaacgtatctgtatgcccctccgggcactctggatcgtacttttctgcgctacggctctgacccggtcgctatctacatgatgctggcggcctccctgctcctgtccccgggtccgctcctggaagagctgggttggcgtggttttgccctgccgcagctcctgaagaaattcgacccgctggctgcagctgtgatcctgggtctgatgtggtgggcatggcatctgccgcgtgatctgccgactctgttctccggtgaaccaggtgcggcttggggcgtgatcgttaaacagttcgtaattatcccgggcttcatcgccggcactattatcgcggtattcgtttgcaacaaactgggcggttccatgtggggtggcgtactgatccatgctattcacaacgaactgggtgtgaacgttaccgcggaatgggctccgacggtggcgggtctgggctggcgcccgtgggatctggtagaattcgctgttgcgatcggcctcgtcctgatctgcggtcgttccctgggcgctgcaagcccggacaacgcacgtctggcgtggggtaacgtgccgcctaaactgccgggcggtgcaaccgataaatccggtgcgaacgcttaagcaacatgtcccggtgggttccgaaaacatttcgggatcaaccagcggtcttactgctggctgcgacggaaatgacggcgaccaaactgttcctggcgctgaccgctgcgatgccgatggcgacgtctcatgttgacccgaaagagctggatgcagtatttgctgatatccgcccggatcagccaggctgtgcctatgctgtagacctgcgtggcaaagttctgtaccagggtggctttggtctggcagatctggcaactcgcgaaccgatcactaccgcgacccgcttcgaactggcaagcaccagcaagcagttcaccgcggctctgatcctgatcctggctcaggaacgccgtctgaagctggccgcgtccatccgtacttacctgccggatctgccaaaagtgtatgacagcgtgaccgtagcggatctcctgcatcacacttccggcatccgcgaatactttgatgcgttccgtacccgcggtgacgatgaaagcaaaccgcactcccgtgaggaagttctggcattcgtcaaagcgcaacgtggcctcgatggtccaccgggccgccgtttctcttatgttaacactaactatttcctcctggccgaaatcgttgaacgtctgactggcaaatcttttccggacgcagcgcgtgaacgtctgttcatcccggcgggtatgaccgaaacccgtgcaactctggatgctacgtccctgattgctggtgacgcccgtggttatcaaattgataaaaacggttcttttgtttccgcggcatggacctggcagggctacggcgatcgtggcgttcgtaccactgtcggcgatctggctctgtggcacggtgcttctctggcagcgactaccggcggtgaagctctgaaggttgctcgcctcgcgaatggtaaactgcgcagcggtcgcagcgtagattacgctggcggtctgttcgtcgacgatcgtcaatccgaacgtgttgtgtcccattctggcctggttgtcggtaaccgcgctatggatgtgctgtacccggattccggtatcggcatctctgttatgtgcaaccgtgatgacatcgccccggcggagcgcgcgcgtaaaattgcgctcctggttaaaccgggtgcgccggatccggcgttcgatcgtgcgattgacccggctgaaatgaaacgtctgggtaaggttggcgacctgcgctctgctccggatggctattaccgcgatcgcctgtacggtcagtatctgattgttgctcaccgtgacggcgaaccgattgtatcttacaacatgcgtgcagaaaaagttacccgccgtcaggatggcatctatcgcgctcgccgtggcgttctcctgagctacgccgtcgcacagtccggtatcaaacgtgtcgtgcagtggaccgaatctggcccgatcccgtaccagtacgtaggtatcggtgcgccggaaacgaaactgttctggccgggccagtaccgtagcgatgagctgggcatcaccgtaaccctgtctcgtgatcagaaaggctggtccctggataccccagcaggtgctgttccgctggcagcggcactggccgacgatctggttggcccggatgctgccttcagcctgcacgccgttggtccgcagatttttaccttccacactgtaaacctgagcggtattgaattccgtcgcctgccgtaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 10 - DNA, 721 bp, Sphingomonas sp. NV3
  CDS (1)..(720): mlrA
attattaccgctgtcaggtctacgttcgagtatccgggagcctatacgctcttactgtttccggccgccccaatgttcgcggccctgatcgcaaccgggatcggctatgggcaagcaggatttcgtgaactgctcagccgctgcgccccgtggcggtcgcctgtttcctggcgtcagggcgttactgtcatagccgtgtgtttccttgcattcttcgcgctcacaggaatcatgtgggttcagacatacctctacgctccgcctggtacgcttgatcgtaccttcctgcgctatggggcagatccggtcgccatttatgtgatgctggcagcatcgctgctactcagccctggcccgctgctggaagaactgggctggcgcggctttgcgctgccgcagctcctcaagaagtttgacccccttaccgcagcggtgatcctcggcatcatgtggtgggcctggcatttgccacgcgacctgccaacactgttctccggcgcccctggcgcggcctggagcgttattgtcaaacaactcgttatcgctcctgggttcattgcgagcaccatcatcgctgtcttcgtatgcaacaagctcggtggatcaatgtgggggggcgtgctcactcacgccatccataacgagctgggagtaaacgtcactgccgaatgggccccaacggtcgcaggcatcgggtggcgcccatgggatctcatcgaat

SEQ ID NO: 11 - protein, 240 aa, Sphingomonas sp. NV3
IITAVRSTFEYPGAYTLLLFPAAPMFAALIATGIGYGQAGFRELLSRCAPWRSPVSWRQGVTVIAVCFLAFFALTGIMWVQTYLYAPPGTLDRTFLRYGADPVAIYVMLAASLLLSPGPLLEELGWRGFALPQLLKKFDPLTAAVILGIMWWAWHLPRDLPTLFSGAPGAAWSVIVKQLVIAPGFIASTIIAVFVCNKLGGSMWGGVLTHAIHNELGVNVTAEWAPTVAGIGWRPWDLIE

SEQ ID NO: 12 - DNA, 1008 bp, Sphingomonas sp. USTB-05
  CDS (1)..(1008): mlrA
atgcgggagtttgtcaaacagcgacctttgctctgcttctatgcgttggcgatcctgatcgctctcgcggcccatgcgctacgcgcgatgagcccgactccgctcggcccgatgttcaagatgctgcaagagacgcacgctcacctcaacattattaccgctgtcaggtccacgttcgagtatccgggagcctatacgcttttgctgtttccggccgccccaatgttcgcggctcttatcgtaaccggtatcggctatgggcgtgcaggatttcgtgaactgctgagccgctgcgccccgtggcgatcgcctgtttcctggcgtcagggcgttactgtcatagctgtgtgtttccttgcgttcttcgcgctcacaggaattatgtgggttcagacattcatctacgctccgcctggtacgcttgatcgcaccttcctgcgctatgggtcagatcccctcgctatttatgcgatgttggcagcatctctgctactcagccctggcccactgctcgaagaactgggctggcgcggctttgcgctgccgcagctcctcaagaagtttgaccctctggccgcagcggtgatcctcggcctcatgtggtgggcttggcatttgccgcgcgacttgccgacgctgttctccggcgaacctggcgcggcctggggcgttatcgtcaagcaattcgttatcattccggggttcattgccggcaccatcatcgctgtcttcgtatgcaacaagctcggcggatcgatgtggggtggcgtgctcattcacgcgatccataacgaactgggcgtaaacgtcactgccgaatgggctccaacggttgcagggcttgggtggcgcccttgggatttggtcgaattcgccgtggccattgggctcgtcctgatttgtggaaggagccttggtgccgcatctcctgacaatgcgcgattggcttggggcaacgtgccgccaaagctgccgggcgtagcgactgacaagtccggcgcgaacgcg

SEQ ID NO: 13 - protein, 336 aa, Sphingomonas sp. USTB-05
MREFVKQRPLLCFYALAILIALAAHALRAMSPTPLGPMFKMLQETHAHLNIITAVRSTFEYPGAYTLLLFPAAPMFAALIVTGIGYGRAGFRELLSRCAPWRSPVSWRQGVTVIAVCFLAFFALTGIMWVQTFIYAPPGTLDRTFLRYGSDPLAIYAMLAASLLLSPGPLLEELGWRGFALPQLLKKFDPLAAAVILGLMWWAWHLPRDLPTLFSGEPGAAWGVIVKQFVIIPGFIAGTIIAVFVCNKLGGSMWGGVLIHAIHNELGVNVTAEWAPTVAGLGWRPWDLVEFAVAIGLVLICGRSLGAASPDNARLAWGNVPPKLPGVATDKSGANA

SEQ ID NO: 14 - DNA, 1011 bp, Novosphingobium sp. THN1
  CDS (1)..(1008): mlrA
atgcgggagtttgtccgacagcggcctttgctgtgcttatatgtgttggcgatactgatcgctctcgcggcccatgcgctacgcgcgatgagcccgacgccgctcgacccgatgttcaagatgctgcaggagacgcacgctcacctcaacattattaccgctgtcaggtctacgttcgagtatccgggagcctatacgctcttactgtttccggccgccccaatgttcgcggccctgatcgcaaccgggatcggctatgggcaagcaggatttcgtgaactgctcagccgctgcgccccgtggcggtcgcctgtttcctggcgtcagggcgttactgtcatagccgtgtgtttccttgcattcttcgcgctcacaggaatcatgtgggttcagacatacctctacgctccgcctggtacgcttgatcgtaccttcctgcgctatgggtcagatccggtcgccatttatgtgatgctggcagcatcgctgctactcagccctggcccgctgctcgaagaactgggctggcgcggctttgcgctgccgcagctcctcaagaagtttgacccccttaccgcagcggtgatcctcggcatcatgtggtgggcctggcatttgccacgcgacctgccaacactgttctccggcgcccctggcgcggcctggagcgttattgtcaaacaactcgtcatcgctcctgggttcattgcgagcaccatcatcgctgtcttcgtatgcaacaagctcggtggatcaatgtgggggggcgtgctcactcacgccatccataacgagctgggagtaaacgtcactgccgaatgggccccaacggtcgcaggcatcgggtggcgcccatgggatctcatcgaatttgccgtggccattggactcgtcctgatttgtgggaggagccttggtgccgcatctcctgacaatgcgcgtttggcctggggcaacgtgccgccaaagcttccgggcggagtgggtgacaagtccggctcgaacgcgtga

SEQ ID NO: 15 - protein, 336 aa, Novosphingobium sp. THN1
MREFVRQRPLLCLYVLAILIALAAHALRAMSPTPLDPMFKMLQETHAHLNIITAVRSTFEYPGAYTLLLFPAAPMFAALIATGIGYGQAGFRELLSRCAPWRSPVSWRQGVTVIAVCFLAFFALTGIMWVQTYLYAPPGTLDRTFLRYGSDPVAIYVMLAASLLLSPGPLLEELGWRGFALPQLLKKFDPLTAAVILGIMWWAWHLPRDLPTLFSGAPGAAWSVIVKQLVIAPGFIASTIIAVFVCNKLGGSMWGGVLTHAIHNELGVNVTAEWAPTVAGIGWRPWDLIEFAVAIGLVLICGRSLGAASPDNARLAWGNVPPKLPGGVGDKSGSNA

SEQ ID NO: 16 - DNA, 561 bp, Acinetobacter lwoffii strain A6
  CDS (1)..(561): mlrA
gacccgatgttcaagatactgcaggagacgcacgctcacctcaacattattaccgctgtcaggtctacgttcgattacccgggagcctatacgcttttgctgtttccggccgccccaatgctcgcggctctgatcgtaaccggtatcggatacgggcgctcaggatttcgtgaactgctcagccgatgcgccccgtggcgatcgcctgtttcctggcgtcagggcgctgccgtcatagccgtgtgtttccttgcgttcttcgcgctcacaggaattatgtgggttcagacatatctctacgctccgcccggcacgcttgatcgcaccttcctgcgctatgggtcagatccggtcgccatttatatgacgctggcagcatcgctgctactcagcccgggcccgctgctcgaagaactgggctggcgcggctttgcgctgccgcagctcctcaagaagtttgaccccctggccgcagcggtgatcctcggcctcatgtggtgggcttggcatttgtcgcgcgacttgccgacgctgttctccagcgatcctggcgcggcctgg

SEQ ID NO: 17 - protein, 187 aa, Acinetobacter lwoffii strain A6
DPMFKILQETHAHLNIITAVRSTFDYPGAYTLLLFPAAPMLAALIVTGIGYGRSGFRELLSRCAPWRSPVSWRQGAAVIAVCFLAFFALTGIMWVQTYLYAPPGTLDRTFLRYGSDPVAIYMTLAASLLLSPGPLLEELGWRGFALPQLLKKFDPLAAAVILGLMWWAWHLSRDLPTLFSSDPGAAW

SEQ ID NO: 18 - DNA, 801 bp, Stenotrophomonas sp. EMS
  CDS (1)..(801): mlrA
atggtcaagatactgcaggagacgcacgctcacctcaacattattaccgctgtcaggtctacgttcgattacccgggagcctatacgcttttgctgtttccggccgccccaatgctcgcggctctgatcgtaaccggtatcggatacgggcgctcaggatttcgtgaactgctcagccgatgcgccccgtggcgatcgcctgtttcctggcgtcagggcgttaccgtcatagccgtgtgtttccttgcgttcttcgcgctcacaggaattatgtgggttcagacatatctctacgctccgcccggcgcgcttgatcgcaccttcctgcgctatgggtcagatccggtcgccatttatatgatgctggcagcatcgctgctactcagcccgggcccgctgctcgaagaactgggctggcgcggctttgcgctgccgcagctcctcaagaagtttgaccccctggccgcagcggtgatcctcggcctcatgtggtgggcttggcatttgccgcgcgacttgccgacgctgttctccggcgatcctggcgcggcctggggcgttatcgtcaagcaattcgttatcattccggggttcattgccagcaccatcatcgctgtcttcgtatgcaacaagctcggcggatcgatgtggggtggcgtgctcattcacgcgatccataacgaactgggcgtaaacgtcactgccgaatgggctccaacggttgcagggcttgggtggcgcccttgggatttggtcgaattcgccgtggccattgggctcgtcctgattgtggggagggag

SEQ ID NO: 19 - protein, 267 aa, Stenotrophomonas sp. EMS
MVKILQETHAHLNIITAVRSTFDYPGAYTLLLFPAAPMLAALIVTGIGYGRSGFRELLSRCAPWRSPVSWRQGVTVIAVCFLAFFALTGIMWVQTYLYAPPGALDRTFLRYGSDPVAIYMMLAASLLLSPGPLLEELGWRGFALPQLLKKFDPLAAAVILGLMWWAWHLPRDLPTLFSGDPGAAWGVIVKQFVIIPGFIASTIIAVFVCNKLGGSMWGGVLIHAIHNELGVNVTAEWAPTVAGLGWRPWDLVEFAVAIGLVLIVGRE

SEQ ID NO: 20 - DNA, 803 bp, Catellibacterium terrae strain A2
  CDS (1)..(801): mlrA
gacccgatgttcaagatactgcaggagacgcacgctcacctcaacattattaccgctgtcaggtctacgctcgattacccgggagcctatacgcttttgctgtttccggccgccccaatgctcgcggctctgatcgtaaccggtatcggatacgggcgctcaggatttcgtgaactgctcagccgatgcgccccgtggcgatcgcctgtttcctggcgtcagggcgttgccgtcatagccgtgtgtttccttgcgttcttcgcgctcacaggaattatgtgggttcagacatatctctacgctccgcccggcacgcttgatcgcaccttcctgcgctatgggtcagatccggtcgccatttatatgacgctggcagcatcgctgctactcagcccgggcccgctgctcgaagaactgggctggcgcggctttgcgctgccgcagctcctcaagaagtttgaccccctggccgcagcggtgatcctcggcctcatgtggtgggcttggcatttgtcgcgcgacttgccgacgctgttctccagcgatcctggcgcggcctggggcgttatcgtcaagcaattcgttatcattccggggttcattgccagcaccatcatcgctgtcttcgtatgcaacaagctcggcggatcgatgtggggtggcgtgctcattcacgcgatccataacgaactgggcgtaaacgtcactgccgaatgggctccaacggttgcagggcttgggtggcgcccttgggatttggtcgaattcgccgtggccattgggctcgtcctgatttgtgggag

SEQ ID NO: 21 - protein, 267 aa, Catellibacterium terrae strain A2
DPMFKILQETHAHLNIITAVRSTLDYPGAYTLLLFPAAPMLAALIVTGIGYGRSGFRELLSRCAPWRSPVSWRQGVAVIAVCFLAFFALTGIMWVQTYLYAPPGTLDRTFLRYGSDPVAIYMTLAASLLLSPGPLLEELGWRGFALPQLLKKFDPLAAAVILGLMWWAWHLSRDLPTLFSSDPGAAWGVIVKQFVIIPGFIASTIIAVFVCNKLGGSMWGGVLIHAIHNELGVNVTAEWAPTVAGLGWRPWDLVEFAVAIGLVLICG

SEQ ID NO: 22 - DNA, 806 bp, Kurthia gibsonii strain A1
  CDS (1)..(804): mlrA
gacccgatgttcaagatactgcaggagacgcacgctcacctcaacattaataccgctgtcaggtctacgttcgattacccgggagcctatacgcttttgctgtttccggccgccccaatgctcgcggctctgatcgtaaccggtatcggatacgggcgctcaggatttcgtgaactgctcagccgatgcgccccgtggcgatcgcctgtttcccggcgtcagggcgttgccgtcatagccgtgtgtttccttgcgttcttcgcgctcacaggaattatgtgggttcagacatatctctacgctccgcccggcacgcttgatcgcaccttcctgcgctatgggtcagatccggtcgccatttatatgacgctggcagcatcgctgctactcagcccgggcccgctgctcgaagaactgggctggcgcggctttgcgctgccgcagctcctcaagaagtttgaccccctggccgcagcggtgatcctcggcctcatgtggtgggcttggcatttgtcgcgcgacttgccgacgctgttctccagcgatcctggcgcggcctggggcgttatcgtcaagcaattcgttatcattccggggttcattgccagcaccatcatcgctgtcttcgtatgcaacaagctcggcggatcgatgtggggtggcgtgctcattcacgcgatccataacgaactgggcgtaaacgtcactgccgaatgggctccaacggttgcagggcttgggtggcgcccttgggatttggtcgaattcgccgtggccattgggctcgtcctgatttgtgggaggag

SEQ ID NO: 23 - protein, 268 aa, Kurthia gibsonii strain A1
DPMFKILQETHAHLNINTAVRSTFDYPGAYTLLLFPAAPMLAALIVTGIGYGRSGFRELLSRCAPWRSPVSRRQGVAVIAVCFLAFFALTGIMWVQTYLYAPPGTLDRTFLRYGSDPVAIYMTLAASLLLSPGPLLEELGWRGFALPQLLKKFDPLAAAVILGLMWWAWHLSRDLPTLFSSDPGAAWGVIVKQFVIIPGFIASTIIAVFVCNKLGGSMWGGVLIHAIHNELGVNVTAEWAPTVAGLGWRPWDLVEFAVAIGLVLICGR

SEQ ID NO: 24 - DNA, 1011 bp, Rhizobium sp. TH
  CDS (1)..(1011): mlrA
atgcgggagtttgtcaggcagcggcctttagtaagcttttatgtgctggcgatcctgatcgctctcgcagccaatgtgctgcgcgcgatggacccgacccccctgggccccatgttcaagatgttgcaagagacgcacgctcacctcaacattgtgacggccatcaggtccacgttcgattatccaacagcctatacgttcctgttgttcccggctgcaccaatgcttgcggccctgattgtgaccggaataggctacggtcgcgcaggatttcgtgagctgctaagccgctgcgccccttggcgagatcctgtttcgtggcggcagggcgtcactgtcattgcggtgtgcttctttgtcttcttcgcgctcaccgggatgatgtgggttcagacctacctatacgctccgtcaggtacgctcgatcgcgcattcctgcgctatgggtcagatccgctctccatttacgcgatgctggcagcatcgcttcttatcagcccgggcccgctgctcgaggagcttggctggcgcgggttcgcactgccccagctgctcaagaagtttgaccccctgacggcggcggtcatcctcggcaccatgtggtgggcctggcatttgccacgcgacttgccggcaatgttctccggcgagcctggtgccctctggggggttatcgtcaagcagttcgttatcgcgcccggaatgatcgccagtacgatcatcgctgtctttgtttgcaacaaactgggtggatcgctgtggggcggattgcttactcacgcgatccataacgagctgggcgtaaacgtaatggccgaatggtcgcccgcggctgcaggactcgggtggcgcccttgggatttcatcgaattcgccgtggccattgggctcgtcctgatttgtggaaggagccttggtgccgcatctcctgacaatgcgcgattggcctggggcaacgtgccgccaaagctgccgggcggagcgactgacaagtccggcgcgaacgcgtga

SEQ ID NO: 25 - protein, 336 aa, Rhizobium sp. TH
MREFVRQRPLVSFYVLAILIALAANVLRAMDPTPLGPMFKMLQETHAHLNIVTAIRSTFDYPTAYTFLLFPAAPMLAALIVTGIGYGRAGFRELLSRCAPWRDPVSWRQGVTVIAVCFFVFFALTGMMWVQTYLYAPSGTLDRAFLRYGSDPLSIYAMLAASLLISPGPLLEELGWRGFALPQLLKKFDPLTAAVILGTMWWAWHLPRDLPAMFSGEPGALWGVIVKQFVIAPGMIASTIIAVFVCNKLGGSLWGGLLTHAIHNELGVNVMAEWSPAAAGLGWRPWDFIEFAVAIGLVLICGRSLGAASPDNARLAWGNVPPKLPGGATDKSGANA

SEQ ID NO: 26 - DNA, 607 bp, Bacillus cereus strain Q1
  CDS (3)..(605): mlrA
gatcgcctgtttcctggcgtcagggcgttgccgtcatagccgtgtgtttccttgcgttcttcgcgctcacaggaattatgtgggctcagacatatctctacgctccgcccggcacgcttgatcgcaccttcctgcgctatgggtcagatccggtcgccatttatatgacgctggcagcatcgctgctactcagcccgggcccgctgctcgaagaactgggctggcgcggctttgcgctgccgcagctcctcaagaagtttgaccccctggccgcagcggtgatcctcggcctcatgtggtgggcttggcatttgtcgcgcgacttgccgacgctgttctccagcgatcctggcgcggcctggggcgttatcgtcaagcaattcgttatcattccggggttcattgccagcaccatcatcgctgtcttcgtatgcaacaagctcggcggatcgatgtgggatggcgtgctcattcacgcgatccataacgaactgggcgtaaacgtcactgccgaatgggctccaacggttgcagggcttgggtggcgcccttgggatttggtcgaattcgccgtggccattgggctcgtcctgatttgtgggaggag

SEQ ID NO: 27 - protein, 201 aa, Bacillus cereus strain Q1
SPVSWRQGVAVIAVCFLAFFALTGIMWAQTYLYAPPGTLDRTFLRYGSDPVAIYMTLAASLLLSPGPLLEELGWRGFALPQLLKKFDPLAAAVILGLMWWAWHLSRDLPTLFSSDPGAAWGVIVKQFVIIPGFIASTIIAVFVCNKLGGSMWDGVLIHAIHNELGVNVTAEWAPTVAGLGWRPWDLVEFAVAIGLVLICGR

SEQ ID NO: 28 - DNA, 1626 bp, Sphingomonas sp. USTB-05
  CDS (1)..(1623): mlrB
atgactgcaacaaagcttttcctggcgctgacggtcgcaatgccaatggcgacttcccatgtcgatccgaaggagctcgatgcggtatttgctgatatccggccggatcaaccgggctgcgcttatgctgtggatctgcgcggcaaggttctctatcagggcggctttggacttgctgatctagccacccgcgagccgatcacacccgcaacacgcttcgaactggcgtcaacatcgaagcagttcacggccgctctcatcctgatcttggtacaggaacgccgacttaaattggcggcatctatccgcacctatctgcctgacctccctaaggtctacgatccggtcacggtcgcggacttgttgcaccacaccagcggcattcgcgagtatttcgatgcattcagggcacgcggagacgatgagagcaaaccccattcccgcgaggaagtgctggccttcgtcaaggcgcaacgcggactcgacggcccacctggccgtcgtttttcctacgtcaacaccaattacttcctgctcgcagaaatcgtggaacgcctaaccggaaagtcatttccggatgctgcgcgggagcggctcttcattccggcgggcatgacggaaactcgcgcaacgcttgatacgaccagtctcgttgcaggtgacgcgcgcggctatcaaatcgacaagaacggtagctttgtctccgcagcctggacctggcaaggctatggcgaccgcggcgtgcggactaatgttggcgatcttgctctttggcatggggcatcgctcgcggcgacaaccggcggtgaggcactcgaagtggcccgcctcgcgaacgggaaactgcgttctggcagatctgtcgattatgccggtgggttgttcgtggatgatcggcaaagcgagcgtgttgtgtcgcattcgggcttggttgtaggcaatcgcgccatggatgtgctgtatccggacagcggcattggtatcagcgtgatgtgcaatcgcgacgatatcgcgccagctgagcgtgcgcgcaaaattgctttgctcgtgaagccgggggcgcccgatccagcatttgatcgcgcaattgatcctgccgaaatgaaacgcctggggaaagttggcgacctgcgctccgcgcctgacggctattatcgcgatcccttgtacggacagtatctcatcgtcgctcaccgagacggtgagccgattgtcagctacaatatgagagctgagaaagtgacgcgccgccaggacggcatctaccgcgcgcgccggggtgttctgctaagctatgcagtcgcacaggttggaatcgagcgtgtcgttcagtggactgaaagtggacccattccgtacgattatgtcggaactggcgcacttgaaaccaagttgtttcggcccggacaatatcgcagcgatgagcttggcatcactgtgaccctgtcaagagatctgaagggatggtcgttggatactcctgcaggtgcagtgcctttagaggctgcgctggcagatgaccttgtgggcccggacgctgcattttcgttgcatgctgttggtcctcaaatctttacatttcacaccgtcaatctgagcgggatagagttcagacggcttccgtag

SEQ ID NO: 29 - protein, 541 aa, Sphingomonas sp. USTB-05
MTATKLFLALTVAMPMATSHVDPKELDAVFADIRPDQPGCAYAVDLRGKVLYQGGFGLADLATREPITPATRFELASTSKQFTAALILILVQERRLKLAASIRTYLPDLPKVYDPVTVADLLHHTSGIREYFDAFRARGDDESKPHSREEVLAFVKAQRGLDGPPGRRFSYVNTNYFLLAEIVERLTGKSFPDAARERLFIPAGMTETRATLDTTSLVAGDARGYQIDKNGSFVSAAWTWQGYGDRGVRTNVGDLALWHGASLAATTGGEALEVARLANGKLRSGRSVDYAGGLFVDDRQSERVVSHSGLVVGNRAMDVLYPDSGIGISVMCNRDDIAPAERARKIALLVKPGAPDPAFDRAIDPAEMKRLGKVGDLRSAPDGYYRDPLYGQYLIVAHRDGEPIVSYNMRAEKVTRRQDGIYRARRGVLLSYAVAQVGIERVVQWTESGPIPYDYVGTGALETKLFRPGQYRSDELGITVTLSRDLKGWSLDTPAGAVPLEAALADDLVGPDAAFSLHAVGPQIFTFHTVNLSGIEFRRLP

SEQ ID NO: 30 - DNA, 1621 bp, Novosphingobium sp. THN1
  CDS (1)..(1620): mlrB
atgactgcaacaaagcttttcctggcgctgacagccgcaatcccaatggcgacttcccatgtcgatccgaaggagctcgatgcggtatttgctgatatccggccggatcaaccgggctgcgcctatgctgtggatctgcgcggcaaggttctctatcagggcggctttggacttgctgatctagccacccgcgagccgatcacacccgcaacacgcttcgaactggcgtcaacatcgaagcagttcacggccgctcttatcctgatcttggcacaggaacgccaacttaaattggcggcatctatccgcacctatctgcctgacctccctaaggtctacgatccggtcacggtcgcggacttgttgcaccacaccagtggcattcgcgagtatttcgatgcattcagggcacgcggagacgatgagagcaaaccccattcccgcgaggaagtgctggccttcgtcaaggcgcaacgcggactcgacggcccacctggccgtcgtttttcctacgttaacaccaattacttcctgctcgcagaaatcgtggaacgcctaaccggaaagtcatttccggatgctgcgcgggaacggctcttcaagccggcgggcatgacggaaactcgcgcaacgcttgatacgaccagtctcattgcaggtgacgcgcgcggctatcaaatcgacaagaacggtagctttgtctccgcagcctgggcctggcaaggctatggcgaccgcggcgtgcggactactgttggcgatcttgctctttggcatggtgcatcgctcgcggcgacaaccggcggtcaggcgctcgaagtggcgcgcctcgcgaacgggaaactgcattctggcagacctgtcgattatgccggtgggttgttcgtggatgatcggcaaagcgagcgtgttgtgtcgcattcgggcttggttgtgggcaatcgcgccatggacgtactttatccggagagcgggattggtatcagcgtgatgtgtaatcgcgacgatatctcgccagctgagcgtgcgcgcaaaattgctttgctcgtgaagcccggggcgcccgatccagcttttgaccgcgcaattgatcctgccgaaatgaaacgcctgggaaaagttggcgacctgcgctccgcgcctgatggctattatcgcgatcccttgtatggacagtatctcatcgtcgctcaccgtgacggtgagccgattgtcagctacaatatgcgagctgagaaagtgacgcgccgccaggacggcatctaccgcgcgcgccgaggtgttctgctgagctatgcagtcatacagagtggaatcaagcgcgtcgttcagtggactgagagtggaccaataccgtaccaatatgttggaactggcgcacctgaaaccaaattgttttggcccggacaatatcgcagcgatgagcttggtattaccgtaaccctgtcacgggatcagaagggatggttgctcgatactccggcaggtgcggtgccgttagctgctgcgatggcagatgaccttgtgggcccgaacgccgcattttcattacatgctgttggtcctcaaatctttacatttcacaccgtcaatctgaacgggatagtgttcaggcggcttc

SEQ ID NO: 31 - protein, 540 aa, Novosphingobium sp. THN1
MTATKLFLALTAAIPMATSHVDPKELDAVFADIRPDQPGCAYAVDLRGKVLYQGGFGLADLATREPITPATRFELASTSKQFTAALILILAQERQLKLAASIRTYLPDLPKVYDPVTVADLLHHTSGIREYFDAFRARGDDESKPHSREEVLAFVKAQRGLDGPPGRRFSYVNTNYFLLAEIVERLTGKSFPDAARERLFKPAGMTETRATLDTTSLIAGDARGYQIDKNGSFVSAAWAWQGYGDRGVRTTVGDLALWHGASLAATTGGQALEVARLANGKLHSGRPVDYAGGLFVDDRQSERVVSHSGLVVGNRAMDVLYPESGIGISVMCNRDDISPAERARKIALLVKPGAPDPAFDRAIDPAEMKRLGKVGDLRSAPDGYYRDPLYGQYLIVAHRDGEPIVSYNMRAEKVTRRQDGIYRARRGVLLSYAVIQSGIKRVVQWTESGPIPYQYVGTGAPETKLFWPGQYRSDELGITVTLSRDQKGWLLDTPAGAVPLAAAMADDLVGPNAAFSLHAVGPQIFTFHTVNLNGIVFRRL

SEQ ID NO: 32 - DNA, 1621 bp, Sphingosinicella microcystinivorans B9
  CDS (1)..(1620): mlrB
atgactgcaacaaagcttttcctggcgctgacggccgcaatcccaatggcgacgccccatgtcgagccgaaggacctcgatgcggtatttgctgatatcaagccggaccaaccgggttgcgcctatgccgtgaacatgcgcggcaaagttctctatcagggtggcttcggtcttgcggacctggccacacgggaaccgattacacccgcaacacgcttcgaactggcgtcaacatcgaagcagttcacggccgctctcatcctgatgttggcacaggaacgccgactaaaattggcggcatcgatccgcacctatctgcctgagctccctaaggtatacgatccggtcacggtcgcggatttgttgcaccacaccagcggcatccgggaatatttcgatgcattcagggcacgcggagacgatgagagcaaaccccattcccgcgaggaagtgctggcatttgtgatggcgcaacggggactcgacggcccacctggccgtcgtttttcttacgttaacaccaattacttcctgctcgcagaaatcgtggagcgcctaaccggaaaaccatttccggatgttgcgcgcgagcggctcttcatgccggcgggcatgaaggaaacgcgcgcaacgcttgatacaacccgtttgattgcaggggatgcacgcggctaccaaatcgccaagaatggtagctttgtcgccgcagcctggacctggcaaggctatggcgaccgcggtgtgcgcaccactgttggcgaccttgctctttggcatggcgcttcgctcgctgctacaaccggcggcaaggcactcgaagtggcccgcctcgtgaacggaaaactgcgctctggcagacctgtcgattatgccggtgggttgttcgtggataatcggcaaggcgagcgtgttgtgtcgcattcgggcttggttgtgggcaaccgggccatggacgtgctctatccggacagcgggatgggtatcagcgtgatgtgcaatcgtgatgatatcgcgccggctgaacgtgcgcgcaaaattgctttgctcgtgaagcccggggcacctgatccggggtttgatcgcgcaattgatcctgccgagatgaagcgcctgggacaaattggtgacctccgcgccgcgcctgatggctactatcgcgatcccttgtacggacagtatctcatcgtcgctcaccgtgacggtgaaccgattgtcagctacaacatgcgagctgagaaagtgacgcgccgccaggatgggatctaccgcgcgcgccggggtgtcctgctaagctatgcaatcgcacgcggcgggcgcgcacgtgttgtccagtggaccgaaagcgggccaatcttatacaactacgtcggaactggcgcacctggagccaagcagttcaggcccggtcgatatcggagcgatgagctcggtgtcactgtaaccctgtcaagggattcgaatggatggacgctgaatactccagcgggtgcggttccgctcgttgctgcgctggcggatgacctggtggccccgaacgccgcattttcattgcatgcaactggtcctcagagttttacatttcacaccgtcaatctgaacagacttatgttccggtggcttc

SEQ ID NO: 33 - protein, 540 aa, Sphingosinicella microcystinivorans B9
MTATKLFLALTAAIPMATPHVEPKDLDAVFADIKPDQPGCAYAVNMRGKVLYQGGFGLADLATREPITPATRFELASTSKQFTAALILMLAQERRLKLAASIRTYLPELPKVYDPVTVADLLHHTSGIREYFDAFRARGDDESKPHSREEVLAFVMAQRGLDGPPGRRFSYVNTNYFLLAEIVERLTGKPFPDVARERLFMPAGMKETRATLDTTRLIAGDARGYQIAKNGSFVAAAWTWQGYGDRGVRTTVGDLALWHGASLAATTGGKALEVARLVNGKLRSGRPVDYAGGLFVDNRQGERVVSHSGLVVGNRAMDVLYPDSGMGISVMCNRDDIAPAERARKIALLVKPGAPDPGFDRAIDPAEMKRLGQIGDLRAAPDGYYRDPLYGQYLIVAHRDGEPIVSYNMRAEKVTRRQDGIYRARRGVLLSYAIARGGRARVVQWTESGPILYNYVGTGAPGAKQFRPGRYRSDELGVTVTLSRDSNGWTLNTPAGAVPLVAALADDLVAPNAAFSLHATGPQSFTFHTVNLNRLMFRWL

SEQ ID NO: 34 - DNA, 1626 bp, Sphingomonas sp. ACM-3962
  CDS (1)..(1623): mlrB
atgactgcaacaaagcttttcctggcgctgacggccgcaatcccaatggcgacgccccaggtcgattcgagggtgctcgatgcggtatttgctgacatcaagccagatgaaccgggctgtgcctatgccgtggatctacgcggcaaggttctttttcagggcggctttggtcttgcggacctgaccactcgcgaaccgattacacccgcgacacgcttcgagttggcgtcaacatcgaagcagtttacagccgccctcattctgatactggcacaggaacgtcgactaagactgacggcatcgatccgcacctatctgcctgacctccctaaggtctacgaaccggtcagcgttgctgatttattgcaccacactagtggcatccgtgaatatttcgatgcattcagggcgcgcggcgaagacgaaagcaaatcccattcccgcgaagaagtgctggcctttatcaaggcgcaaagagggctcgacggccctcctggccaccgtttttcgtacgtaaacaccaattacttcctgcttgcggagatcgtggagcgcctaattagaaaaccatttccggatggtgcgcgcgagctgctcttcatacctgcgggcatgaaggaaacgcgcgcaacacttgatacgaccagtctgctcgcaggggacgcacacggttatcaagtcaccaaggacgggaaatttgccgaagccgtctgggcctggcaaggctatggcgaccgcggcgtgcggactactgttggcgatcttgctgtttggcatggcgcatcgctcgcggcgacaaccggcggtcaggcactcgaagtggcccgccttgcgaacggaaaactgcgttctggcaggcctgtcgattatgccggtggattgttcgtcgacgatcggcaaagcgagcgtgttgtttcgcattcgggcttggttgtgggcaatcgagccatggacgtaatgtttccggacagcggacttggtatcagcgtgatgtgtaatcgcgacgatatctcgccagctgagcgtgcgcgcaaaattgctttgctcgtgaagcccggggcgcccgattcagcttttgatcgcgcgattgatcctgccgaaatgaaacgcttgggaagaattggcgacctgcgctccgcacctgacggctattatcgcgatcccttgtacggacagtatctcatcgtcgatcaccgtggcggtgagccgatcgtcagctacaatatgcgagctgagaaagtgacgcgccgccaggacggcatctaccgcgcgcgccggggtattctgctgagctatgcagtcgcacagggtggacccaagcgtgtcgttcagtggactgagagtggaccaatttcgtacgaatatgtcggaattggcgcacctgaagccaagttgttttggcccggacaatatcacagcgatgagcttggtattaccgtaaccctgtcaagggatacgaagggttggtcgctggatactccggcaggggcggtgccgttagctgctgcgctggaagatgaccttgtgggcccgaacgccgcattttcattacatgctgttggtcctcaaatctttacatttcacaccgtaaatctgaacgggatagtgttcaggcggcttcgataa

SEQ ID NO: 35 - protein, 541 aa, Sphingomonas sp. ACM-3962
MTATKLFLALTAAIPMATPQVDSRVLDAVFADIKPDEPGCAYAVDLRGKVLFQGGFGLADLTTREPITPATRFELASTSKQFTAALILILAQERRLRLTASIRTYLPDLPKVYEPVSVADLLHHTSGIREYFDAFRARGEDESKSHSREEVLAFIKAQRGLDGPPGHRFSYVNTNYFLLAEIVERLIRKPFPDGARELLFIPAGMKETRATLDTTSLLAGDAHGYQVTKDGKFAEAVWAWQGYGDRGVRTTVGDLAVWHGASLAATTGGQALEVARLANGKLRSGRPVDYAGGLFVDDRQSERVVSHSGLVVGNRAMDVMFPDSGLGISVMCNRDDISPAERARKIALLVKPGAPDSAFDRAIDPAEMKRLGRIGDLRSAPDGYYRDPLYGQYLIVDHRGGEPIVSYNMRAEKVTRRQDGIYRARRGILLSYAVAQGGPKRVVQWTESGPISYEYVGIGAPEAKLFWPGQYHSDELGITVTLSRDTKGWSLDTPAGAVPLAAALEDDLVGPNAAFSLHAVGPQIFTFHTVNLNGIVFRRLR